(19) United States
(12) Patent Application Publication
(10) Pub. No.: US 2007/0160561 A1
(43) Pub. Date: Jul. 12, 2007

(54) AMPHIPHILIC STAR BLOCK COPOLYMERS

(76) Inventors: Lahoussine Ouali, Vetraz-Monthoux (FR); Andreas Herrmann, Veyrier (CH); Celine Ternat, Palezieux (CH); Christopher John George Plummer, Echandens-Denges (CH); Harm-Anton Klok, Saint-Sulpice (CH); Georg Kreutzer, Dubendorf (CH); Jan-Anders E. Manson, Chexbres (CH); Horst Sommer, Ornex (FR); Maria Ines Velazco, Geneva (CH)

Correspondence Address: WINSTON & STRAWN LLP;PATENT DEPARTMENT, 1700 K STREET, N.W., WASHINGTON, DC, 20006, US

(21) Appl. No.: 11/690,074

(22) Filed: Mar. 22, 2007

Related U.S. Application Data
(63) Continuation of application No. PCT/IB05/03023, filed on Oct. 10, 2005.

(30) Foreign Application Priority Data
Oct. 8, 2004 (EP) 04 104 955.2

(51) Int. Cl. A61K 8/81; C08L 61/00; A01N 25/00

(52) U.S. Cl. 424/70.16; 525/143; 424/405; 977/906

(57) ABSTRACT

The present invention relates to a basically spherical hyperbranched block copolymer having an internal hydrophobic block and an external hydrophilic block. Within the spherical copolymer, the hydrophobic block constitutes a hydrophobic layer, suitable to associate or encapsulate hydrophobic bioactive agents, while the hydrophilic block provides an outer layer, which is suitable to render the copolymer soluble or dispersible in aqueous solutions. Also claimed is a method for preparing the copolymer, which is suitable to encapsulate fragrances, flavours, drugs, agrochemicals, for example.

### CROSS-REFERENCE TO RELATED APPLICATIONS

This application is a continuation of International application PCT/IB2005/003023 filed on Oct. 10, 2005, the entire content of which is expressly incorporated herein by reference thereto.

### TECHNICAL FIELD

The present invention relates to block copolymers comprising a multifunctional core, a hydrophobic block and a hydrophilic block. The present invention further relates to nano-capsules, formed of the block copolymers, and a process for manufacturing a block copolymer.

### BACKGROUND OF THE INVENTION

The delivery of functional agents, molecules, ingredients, or compositions such as flavours, fragrances, pharmaceuticals, agrochemicals such as herbicides, fungicides or pesticides, dyes, and many others is an important issue for nearly all applied sciences. Without the stabilisation of a concentrated, easily transportable and processible form of the functional agent delivery becomes unreliable and the agent will only rarely exhibit its beneficial properties at the predetermined place and time. Indeed, effective encapsulation is required in a wide range of applications in order to protect sensitive additives from degradation and to control their release and hence optimise their performance according to the requirements of the application.

Thus, encapsulation is key when it comes to the delivery of stabilised functional agents, and many different encapsulation technologies and systems have been developed so far. A particular group of encapsulation systems, micro- or nanocapsules is concerned with the problem of providing particles which comprise hydrophobic functional agents, such as fragrances or flavours, but which are dispersible or soluble in an aqueous environment, such as in the aqueous phase of an emulsion, for example a shampoo, lotion or shower-gel.

A biodegradable copolymer composition comprising a polysaccharide backbone and amphiphilic diblock copolymers is disclosed in EP 04101930.8. However, it would be an advantage to have further delivery systems, possibly capable of encapsulating higher loads of bioactive molecules. In addition, it would be an advantage to have capsules with more spherical shapes.

Polymeric micro- and nanocapsules formed of a spherical single molecule are the subject of EP 1 443 058 A1. These capsules are formed of a single cross-linked hydrophobic polymer, which has been chemically modified by means of a chemical agent so as to comprise hydrophilic moieties at its surface. The chemical modification is made by adding a carboxylic acid, a quarternary ammonium, a hydroxy, sulfonate or yet thiol moiety at the surface of the particle. However, these capsules are prepared by emulsion-polymerisation, which means that the encapsulate occupies the entire centre of the capsules, while the polymer is located in the form of a shell around the encapsulate. Such capsules are not very resistant to mechanical stress.

In U.S. Pat. Nos. 6,723,789 B1, 6,552,146 B1, and 6,476,124 B1, block copolymers having a star structure of the formula A-[(M1)ₚ₁-(M2)ₚ₂ . . . (Mi)ₚⱼ]ₙ are disclosed, basically for cosmetic applications (nails, eyes lashes, eyebrows and hair). However, these copolymers are not amphiphilic and are not suitable as encapsulation systems.

Hyperbranched amphiphilic polymeric additives are disclosed in WO 01/58987 A2, however, not for the encapsulation of bioactive agents. EP 0 858 797 A1, in contrast, deals with dendritic polmers carrying a terminal amino function, for treating axillary malodours. A lipid core containing lipophilic active principles and a water-insoluble continuous polymeric envelope, on the other hand, are the subject of U.S. Pat. No. 6,379,683 B1.

A further reference in the field of encapsulation by polymers is WO 02/26867 A2, where a specific family of dendrimers are used for the delivery of drugs. These molecules are, however, relatively small, thus limiting the amount of encapsulate to be delivered.

In view of the prior art it becomes apparent that further encapsulation systems are needed, especially those that are suitable to encapsulate lipophilic, optionally volatile bioactive agents in an aqueous environment. Generally, good solubility or dispersibility of the capsule in water means that sedimentation as well as floating of the capsules on the surface of an aqueous liquid is to be avoided. Further important parameters are the amount of bioactive agent that can be loaded or encapsulated per weight unit of capsule, the ability to control its release as well as the physical stability of the capsule.

The present invention is concerned with addressing and resolving these problems.

### SUMMARY OF THE INVENTION

Remarkably, a basically spherical block copolymer could be provided, which contains a multifunctional centre, a hydrophobic block, polymerised onto the centre or an adequate linker molecule, and, proximally, a hydrophilic block, enabling good solubility or dispersibility in aqueous liquids. Thanks to the hydrophobic block, the copolymer contains a relatively large sphere or layer, in which hydrophobic (bio)active agents are easily associated or bound.

Accordingly, the present invention provides, in a first aspect, a block copolymer compound comprising the general formula (I) 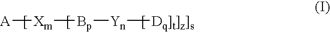
wherein:

A is a core having s functionalities; s multiplied by z defines the number of arms of the copolymer, whereby the product of s*z >6;

Xm and Yn are, independently of each other, a linear or branched linker moiety with m or n, independently of each other, being 0 or 1, which is, once grafted to the core, suitable as a starting point for at least one polymerisation reaction;

z and t are the number of branchings provided by each of the linker moieties X and Y, respectively, with z and t being, independently, in the range of 1-10;

B is a polymerised moiety having a calculated Hansen solubility parameter of ≦25, which is covalently linked to a functionality of A or to a functionality of X, with p being the average number of polymerised B moieties, p is in the range of 3-300;

D is a polymerised moiety having a Hansen solubility parameter of >25 with q being the average number of polymerised D moieties, q is in the range of 3-300.

In a second aspect, the present invention provides a nano-capsule essentially consisting of the block polymer according to the invention.

In a third aspect, the present invention provides a block copolymer, suitable for encapsulation of hydrophobic bioactive molecules, the block copolymer comprising, in this order,

a central, lipophilic or hydrophilic, multifunctional core (A),

a lipophilic block (B), and,

a hydrophilic block (D);

and, optionally, one or more linker molecules between the core and the lipophilic block (X) and/or between the lipophilic block and the hydrophilic block (Y).

In a fourth aspect, the present invention provides a process manufacturing a block copolymer comprising the steps of

providing a core (A) having s functionalities, with s>5,

optionally, linking the functionalities of the core to a linker moiety (X),

polymerising a hydrophobic block (B) onto the functionality of the core, or, if present onto the linker moiety (X),

optionally, linking a further linker moiety (X) onto the hydrophobic block (B), and

polymerising a hydrophilic block (D) onto the functionality of the hydrophobic block (B) or of the further linker moiety (X), or, alternatively, polymerising a second hydrophobic block onto the first block or onto the further linker moiety, followed by chemically transforming the second hydrophobic block into a hydrophilic block (D).

The present invention further provides the use of the block copolymer according as disclosed above for encapsulating and/or associating at least one lipophilic functional agent. Furthermore, the invention provides a method for encapsulating and/or associating substantially as set out in the claims. The invention also provides a perfumed product comprising the block copolymer of the invention.

### BRIEF DESCRIPTION OF THE DRAWING FIGURES

In the Figures,

FIG. 1 illustrates an example for the structure of the copolymer of the present invention by indicating in more detail one of (here: s=11) branches of the copolymer of the present invention. According to this structure, linker molecules X and Y are present and both have t, z=3 functionalities. Dashed lines are used to simplify the figure and indicate where branches of the polymer are extending following the principle given in formula (I) above.

FIG. 2 represents the ¹H-NMR quantification data obtained for the encapsulation of fragrances into the amphiphilic H40-X-(Pn-BuMA)₃₀-(PPEGMA)₃₂ star block copolymer. The curves show a linear correlation between the amount of polymer in solution and the quantity of fragrance detected, thus demonstrating the successful encapsulation of the fragrance molecules in the polymer.

FIG. 3 shows the comparison of the amount of benzyl acetate encapsulated in H40-X-(PPEGMA)₄₀ and H40-X-(Pn-BuMA)₃₀-(PPEGMA)₃₂, respectively. The curves give evidence for the advantage of having star block copolymers with a hydrophobic block and a hydrophilic block.

FIG. 4 shows the increased retention of a fragrance compound (citral) in the copolymer or the nano-capsules of the present invention if compared to a reference sample of pure, not encapsulated citral. It can be seen from the figure that the release of citral in the nano-capules of the present invention over 10 h is strongly slowed down if compared to non-encapsulated citral.

FIG. 5 represents a thermogravimetric analysis illustrating the evaporation (weight in % relative to the initial weight at the beginning of the experiment as a function of time in min) of geraniol alone, geraniol in the presence of Boltorn® H40 HBP and geraniol in the presence of the amphiphilic star block copolymer H40-(PCL)₁₀-Y-(PAA)₇₀.

FIG. 6 shows the evaporation profile of allyl 3-cyclohexylpropanoate in the presence (-▪-) or absence (-∘-) of amphiphilic star block copolymer H40-(PCL)₁₀-Y-(PAA)₇₀ as measured by dynamic headspace analysis of a model perfume.

FIG. 7 shows the headspace concentrations measured over time for the release of allyl 3-cyclohexylpropanoate in the presence (-▪-) or absence (-∘-) of amphiphilic star block copolymer H40-(PCL)₁₀-Y-(PAA)₇₀ as measured by dynamic headspace analysis in a fabric softener application.

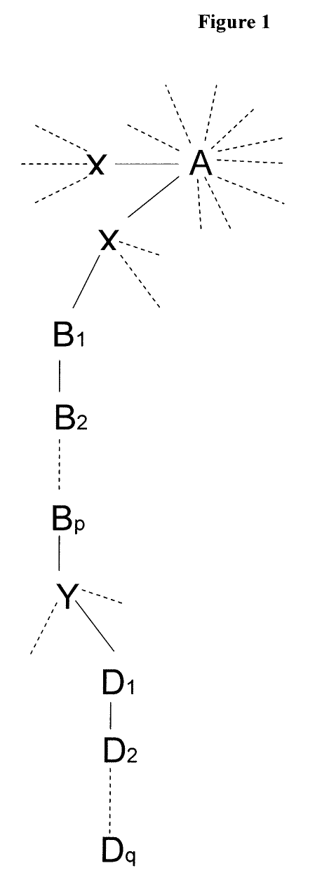

### DETAILED DESCRIPTION OF THE PREFERRED EMBODIMENTS

Within the context of this specification the word “comprises” is taken to mean “includes, among other things”. It is not intended to be construed as “consists only of”.

In the context of the present invention, percentages are percentages by weight of dry matter, unless otherwise indicated. Similarly, if proportions are indicated as parts, parts of weight of dry matter are meant.

The terms “average” or “mean” as used, for example in the expression “average degree of polymerisation” or “mean diameter” refers to the arithmetic mean.

The term “functionality” refers to a functional group of a compound suitable to be covalently linked to a further compound or suitable to mediate a covalent binding reaction, be it a to linker compound or be it a moiety that can be polymerised. Suitable functionalities in the above sense may be selected from, for example, —OH, —NH₂, —CN, —NCO, —COOH, —X, X being a halogen, preferably Cl and/or Br). “Multifunctional” thus means that a specific compound has several, for example s, functionalities.

The Hansen solubility parameter of ≦22 or >22, for example, is a measurement for determining the hydrophilicity/hydrophobicity of a polymerised moiety and is calculated, for the purpose of the present invention, by the software Molecular Modeling Pro, version 5.22, commercialised by Norgwyn Montgomery Software Inc, ©2003. The dimension of the Hansen solubility parameter is (MPa)^{1/2}, which is valid throughout the present document.

For unambiguously determining the Hansen solubility parameter for the purpose of the present invention, it is herewith determined that a number of 8 polymerised monomeric units with unrepeated terminal endings replaced by H— are taken to calculate the parameter by means of the above-indicated software. For example, for a polymer comprising tert-butyl acrylate as monomeric moieties the molecule below is used to calculate the Hansen solubility parameter. The value obtained with Molecular Modeling Pro is 19.84. 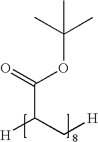
The value of 25 for the Hansen solubility parameter was found by the inventors to be a value for the hydrophilic block (D) above which the copolymer of the present invention becomes dispersible or soluble in water.

The term “lipophilic functional agent” or “hydrophobic functional agent” refers to molecules having a calculated octanol/water partition coefficient (clogP) of ≧1, preferably ≧0, more preferably ≧2, most preferably ≧4. This parameter is calculated by the software T. Suzuki, 1992, CHEMICALC 2, QCPE Program No 608, Department of chemistry, Indiana University. See also T. J. Suzuki, Y. Kudo, J. Comput.-Aided Mol. Design (1990), 4, 155-198.

In an aspect of the present invention, a nanocapsule essentially consisting of the block polymer according to the invention is provided. The nanocapsule is formed by the copolymer of the present invention due to the multifunctionality of the core (s>5), optionally further branched arms extending from the core (A). The number of arms of the block copolymer is determined by the multiplication product of the number of functionalities on the core (s) and the numbers (z) of potential branching at the linker moiety X. If there is no linker moiety X (m=0), z equals 1 as the following diblock is directly linked to the functionalities s of the core. In this case, the block copolymer comprises s arms. According to another example, if there is a linker X, comprising a single branching, z becomes 2 and the number of total arms will be 2 times the number of functionalities of the core.

Preferably, the number of arms (s*z) is >8. According to an embodiment, the block copolymer of the present invention comprises >12 arm (s*z). More preferably, the number of arms is >15, even more preferably >20, and most preferably >25. According to preferred embodiments, the number of arms is >30, >40 or even >50. The more arms are present, the larger the block copolymer of the invention, and the larger the hydrophobic compartment within the copolymer, enabling more lipophilic agents being associated within the block copolymer of the present invention. The block copolymer preferably has ≦100 arms, more preferably ≦800 arms, and even more preferably ≦70 arms and most preferably ≦64 arms. Analytically, the number of arms may be deduced from the number of functionalities s of the core A.

Each arm of the copolymer of the present invention is defined by the presence of at least one hydrophobic block B and at least one hydrophilic block D. Block B forms a hydrophobic layer within the overall spherical shape of the copolymer of the present invention. The arms further comprise more distally at least one relatively more hydrophilic block D, forming the outer layer of the capsule. The outer layer is suitable to render the capsule soluble and/or dispersible in water.

Each block, be it B or D, is defined as a non-branched, linear polymer. Branching of the copolymer of the invention may occur at the positions X or Y, which are the optional linkers, separating different blocks. Branchings may also be present in the core A.

The present invention also provides a delivery system for functional agents, the delivery system comprising the nanocapsules of the present invention.

Preferably, the compound of the present invention is a star block copolymer, more preferably it is an amphiphilic star block copolymer, most preferably it is a multiple-arm amphiphilic star block copolymer.

In an embodiment, the copolymer of the present invention has a mean diameter of 2-150 nm. Preferably, it has a diameter of 10-100 nm, more preferably 15-80 nm.

In an embodiment, the compolymer of the invention has a molecular weight of Mn>100,000 g/mol. Preferably, the Mn is >120,000, for example >140,000, more preferably it is >160,000, for example >180,000. Even more preferably Mn is >200,000, for example >250,000, and most preferably it has a molecular weight of Mn >300,000 g/mol.

The present invention provides a copolymer of the general formula (I) 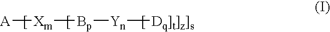
in which A represents a core. The core may be any molecule providing functionalities suitable as a starting point (also called initiator) for attaching a linker molecule or as a starting point for a polymerisation reaction. The core thus preferably carries functionalities as defined above on the surface (s>5), preferably it carries more than 10 functionalities (s>10), more preferably more than 15 (s>15), more than 20 (s>20), more than 30 (s>30) and most preferably it carries more than 40 (s>40). For example, it may have more than 100 (s>100) functionalities as defined above on its surface. The core may thus be a polymer, for example a hyperbranched polymer, a dendrimer or a multifunctional low molecular weight molecule. A low molecular weight molecule in this sense is a monodisperse molecule having a fixed or constant molecular weight in the range of 500-1500.

In a preferred embodiment of the present invention, the core A is a hyperbranched or a dendritic polymer.

Suitable cores which may be used in the sense of the invention are poly(aryl ethers) as for example those disclosed in C. Hawker and J. M. J. Fréchet, “A New Convergent Approach to Monodisperse Dendritic Macromolecules”, J. Chem. Soc., Chem Commun. 1990, 1010-1013, once functionalised at their surface, or those described in Y. Zhao, et al., “Synthesis of novel dendrimer-like star block copolymers with definite numbers of arms by combination of ROP and ATRP”, Chem. Commun, 2004, 1608-1609 having —OH groups at their surface.

Another class of suitable hyperbranched or dendritic cores (A) according to the present invention are poly(amidoamines) (PAMAM), having a —NH₂ function at the surface, which are commercially obtainable from Dendritech® Inc., USA.

As alternative, equally suitable core molecules with —NH₂ functionalities at the surface, one may cite poly(ethylene imines), commercialised by Hyperpolymers GmbH, Germany, or poly(propylene imines), Astramol™, commercialised by DSM, The Netherlands.

A further group of suitable core structures (A) are poly(aminoesters), as disclosed in J. Park and co-workers, “Cationic Hyperbranched Poly(amino ester): A Novel Class of DNA Condensing Molecule with Cationic Surface, Biodegradable Three-Dimensional Structure, and Tertiary Amine Groups in the Interior”, J. Am. Chem. Soc. 2001, 123, 2460-2461.

Other cores (A) that may be used according to the present invention, are polyurethanes, having —OH and/or —NCO functions at their surface, as disclosed in DE 195 24 045 A1, or polyglycerols, also having —OH functions at the surface and being commercialised by Hyperpolymers GmbH, Germany.

The above-given, purely exemplary list of suitable core substances in the copolymer of the present invention may be further completed by polyesters, having an —OH function at their surface, as for example those disclosed in WO 01/46296 A1 or, preferably, those marketed under the brand name Boltorn™ by Perstorp, Sweden, especially the products H20, H30 and H40. In an embodiment of the present invention, the core (A) is a hyperbranched polyester.

The calix[8]arenas-based initiator disclosed in Example 1 of U.S. Pat. No. 6,476,124 B1 may also be used as a core (A) for the purpose of the present invention. This is an example for a monodisperse low molecular weight molecule.

Basically, any hyperbranched polymer or dendrimer having functionalities on its surface can be selected. The skilled person can select suitable cores based on technical skills, for example from the textbook “Dendrimers and Dendrons” of Newkome et al, Wiley-VCH Verlag GmbH, 2001 or from other textbooks.

The present invention provides a block copolymer comprising a block Bₚ and a block D_{q}, which are polymerised moieties having a calculated Hansen solubility parameter of ≦25 (block Bₚ) or >25 (block D_{q}) when polymerised, respectively.

Block B is covalently linked to a functionality at the surface of A or, if present, to a functionality of X, with p being the number of polymerised B moieties. The value of p is in the range of 3-300. Preferably, p is in the range of 5-200, for example 10-100, more preferably 8-60, even more preferably 9-40, most preferably 10-35.

Block B is also referred to as the hydrophobic or lipophilic block, because it has the purpose of encapsulating, absorbing or associating lipophilic or hydrophobic bioactive molecules.

For the purpose of the present invention, the calculated Hansen solubility parameter of ≦25 encompasses polymers that can associate to or encapsulate lipophilic compounds.

Regarding the actual structure of the polymerised moiety B it is clear that any structure fulfilling the criteria of Hansen solubility parameter of ≦25 can be used, because only this solubility parameter determines the ability of the block copolymer to encapsulate and/or associate hydrophobic bioactive agents. In order to avoid any doubt it is clarified that the solubility parameter needs to be calculated on the basis of the moiety that is part of the final block copolymer. It is thus possible to chemically modify one of the blocks, for example B and/or D in an additional step following polymerisation, in order to obtain the required value of the Hansen solubility parameter.

Generally, monomeric moieties for block B may be selected from the prior art. An illustrative list of suitable monomers in case of atom transfer radical polymerisation (ATRP) is given in U.S. Pat. No. 6,692,733, col. 4, line 12-col. 6, line 38, where the general structure according to the formula (II) 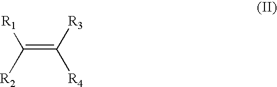
is given, in which R₁, R₂, R₃ and R₄ are defined in the above-indicated text position, which is explicitly incorporated herein by reference. These monomeric moieties are suitable to be used in other types of polymerisation, as the skilled person will know. Of course, from possible monomers according to the above structure, only those fulfilling the requirement of the Hansen solubility parameter of the present invention may be selected.

Preferably, monomers for preparing block B of the present invention may selected from monomers having the formula (III) 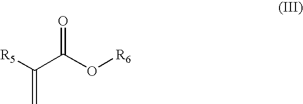
with R₅ being H or CH₃ and R₆ being CₙH₂ₙ₊₁, linear or branched, with n=1-10.

Examples of this type of monomer are methyl methacrylate, methyl acrylate, propyl methacrylate, propyl acrylate, butyl methacrylate, butyl acrylate, tert-butyl methacrylate, tert-butyl acrylate, pentyl methacrylate, pentyl acrylate, hexyl methacrylate, hexyl acrylate.

Another suitable monomeric moiety typically suitable in the preparation of block B of the present invention has the formula (IV) 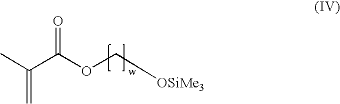
with w being 1 or 2.

A further monomeric moiety suitable for preparing the hydrophobic block B is vinyl acetate.

Alkyl styrenes are still further monomeric moieties for preparing block B. The alkyl preferably is a C₁ to C₅ linear or branched alkyl group. Styrene as such, devoid of an alkyl residue, may also be used.

Specifically referring to ring-opening polymerisation (ROP), the monomer ε-caprolactone could be polymerised directly on a —OH group of the hyperbranched core (A) or of the optional linker (X) and could thus preferably be employed as a moiety in the preparation of block B according to the present invention. Generally, block B may be a polymer selected from the group consisting of polylactides, polycaprolactone, polypropylene glycol and polyanhydrides.

Therefore, in an embodiment of the present invention, block B of the copolymer of the present invention is selected from the group consisting of poly(methyl methacrylate), poly(methyl acrylate), poly(n-butyl methacrylate), poly(n-butyl acrylate), polylactides, polycaprolactone such as poly(ε-caprolactone), polypropylene glycol, polyanhydrides, polysiloxanes, polyphosphazenes, polyazolines and combinations thereof.

The block copolymer compound of the present invention comprises a block D_{q}, which is a polymerised moiety having a Hansen solubility parameter of >25 with q being the number of polymerised D moieties. The value of q is in the range of 3-300. Preferably, q is in the range of 5-200, more preferably 10-150, for example 10-100, even more preferably 15-80, for example 15-70, and most preferably 25-75, for example 30-73.

Block D is generally referred to as the hydrophilic or lipophobic block, because the purpose of this block is to render the copolymer soluble or dispersible in water. Regarding the Hansen solubility parameter, the comments made above apply, with the value of the parameter being the important difference between blocks B and D.

The monomeric moieties of block D may thus be selected from any moiety giving rise to a Hansen solubility parameter of >25 when polymerised. Block D may be neutral or it may carry positive and/or negative charges.

Suitable moieties for polymerising block D or the copolymer of the present invention may be selected from the compounds covered by the formula V below 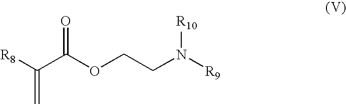
in which R₈, R₉ and R₁₀ are selected, independently of each other, from H, —CH₃, —CH₂—CH₃. Examples of these compounds include diethyl amino methacrylate, diethyl amino acrylate, dimethyl amino methacrylate, dimethyl amino acrylate. The compounds of formula (VI) may be further modified after polymerization by quaternisation of the N-atom, to obtain a positively charged moiety.

Another example of a monomeric moiety useful in the preparation of block D of the present invention is tert-butyl methacrylate or tert-butyl acrylate, which was already mentioned above in the context of suitable block B moieties. For fulfilling the requirements of the Hansen solubility parameter of >25, however, the block D being constituted of tert-butyl methacrylate or acrylate has to be further modified to render it more polar or hydrophilic. This can easily be done by a hydrolysis of the tert-butyl-group subsequent to the polymerisation, leaving an —OH group at the place of the tert-butyl ester. The hydrolysis may be incomplete, according to the reactants and the conditions selected. The degree of hydrolysis required to solubilise the copolymer of the invention in water may be determined by the skilled person and will depend on different factors, such as the DP of block B and D, the nature characteristics of the polymer, and so forth.

In addition, the hydrophobic moieties of block D may be selected from compounds of the formula (VI) 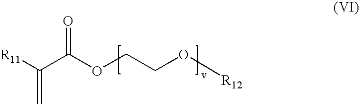
with R₁₁ and R₁₂ being, independently of each other, H or —CH₃, and v being 1-10.

As a further example, hydroxy ethyl methyacrylate and hydroxy ethyl acrylate may also be used as monomeric moieties in the preparation of block D.

As a still further example, vinyl acetate may be used as a monomeric unit, if it is hydrolysed after polymerisation in order to become a hydrophilic moiety of block D. In this case, block D will be a poly(vinylalcohol).

As described above in the context of block B, a monomeric unit for block D_{q} may be selected from formula (II), as long as the Hansen solubility requirement for block D is fulfilled. These monomeric units are particularly suitable for ATRP.

In an embodiment of the present invention, D_{q} of the copolymer of the present invention is selected from the group consisting of poly(methacrylic acid), poly(acrylic acid), poly(dimethyl aminoethyl methacrylate), poly(trimethylaminoethyl methacrylate), poly(trimethylaminoethyl acrylate), poly(trimethylammoniumethyl methacrylate salts), poly(hydroxyethyl methacrylate), poly(methylether diethyleneglycol methacrylate), poly(ethylene oxide), poly(vinylpyrrolidone), poly(polyethylene glycol acrylate), poly(polyethylene glycol methacrylate), polyaminoacids, polyacrylonitriles, poly(ethylene imine), and, polyoxazoline, and combinations thereof.

Both blocks, the hydrophobic block B and/or the hydrophilic block D may further comprise so-called AB*-type monomers, which can be used to introduce a branching within block B and/or D by self condensing vinyl co-polymerisation. An example of such a monomer is 2-(bromoisobutyryloxy)ethylmethacrylate, other examples are disclosed in H. Mori, A. H. E. Müller, Adv. Polym. Sci. 2003, 228, 1-37. In this way, the shells of the nanocapsule of the present invention will be comprised of further branched polymers and will thus be denser. AB*-type monomers may be added to the hydrophobic/hydrophilic monomers of block B and/or D during preparation of the block in amounts of 0.5 to 5 mol-%.

Principally, any type of polymerisation can be employed to polymerise the hydrophobic block B or hydrophilic block D. Examples of possible polymerisation methods are anionic and/or, cationic polymerisation, polyaddition, polycondensation, free radical polymerisation, for example controlled free radical polymerisations, the latter including atom transfer radical polymerisation (ATRP), and reversible addition-fragmentation chain transfer (RAFT), and, stable free radical polymerisation (SFRP), such as nitroxide-mediated polymerisation, and, as a further type of polymerisation: ring opening polymerisation (ROP).

Preferably, the copolymer of the present invention is made by ATRP, RAFT, ROP, or two of these.

Block B and D of the copolymer of the present invention may be prepared using the same or different types of polymerisation.

The block copolymer of the present invention optionally comprises, covalently bound to the core, and/or to the hydrophobic block B, a linear or branched linker compound (X and/or Y). The linker compound may be used to provide a suitable starting point, also called initiator, for polymerisation. If a linker X, for example, provides two starting points, it is a branched linker moiety with z being 2. Preferably, z and t are, independently, in the range of 1-5.

The linker can be multivalent, that is, it may be branched in a way that it serves as initiator for more than one polymerisation reaction per linker. If the linker is multivalent or branched, the value of t and/or z in the compound of formula (I) will become >1, that is t and/or z will correspond to the number of branches initiated by the linker.

Depending on the type of polymerisation selected for polymerising blocks B and/or D, the skilled person is capable of selecting linker compounds (X and/or Y) suitable as initiator. An exemplary list of suitable linkers is given below.

For ATRP the linker may be a secondary C₂-C₁₅ alkyl halogenide, preferably a secondary C₃-C₁₀ alky halogenide. Preferably, the halogenide is selected from the group consisting of chloride, iodide and/or bromide. Preferably, the halogenide is a bromide. Other suitable linkers for ATRP are benzyl halides, haloesters, haloketones, halonitriles, sulfonyl halides, allyl halides, haloamides, for example.

For example, the linker compound is characterised by the presence of at least one radically transferable atom or group. For example, molecules of the general formula (VII) 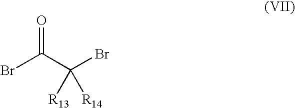
with R₁₃ and R₁₄ being, independently of each other, selected from Bromine, H, or an optionally further substituted C₁-C₃ alkyl residue, preferably a methyl group.

Examples for such and other linkers are 2-bromoisobutyryl bromide, 2-bromopropionyl bromide, in which the bromine atom(s) at the C2 position is (are) radically transferred. Of course, the corresponding chlorides or iodides of the above compounds are equally suitable. An example of a branched linker is 2,2-dibromopropionyl bromide.

Other examples for linkers (X, Y) suitable in ATRP are disclosed in K. Matjaszewski, J. Xia, Chem. Rev. 2001, 101, 2921-2990 and in M. Kamigaito, T. Ando, M. Sawamoto, Chem. Rev. 2001, 101, 3689-3745.

Linkers suitable for RAFT are alkyl iodides, xanthates (see M. H. Stenzel, L. Cummins, E. Roberts, T. P. Davis, P. Vana, C. Barner-Kowollik, Macromol. Chem. Phys. 2003, 204, 1160-1168) and dithiocarbamates (WO 9935177), for example.

Linkers suitable for SFRP are nitroxides and alkoxy amines (for nitroxide mediated polymerisation, see C. J. Hawker, A. W. Bosman, E. Harth, Chem Rev. 2001, 101, 3661-3688), borinates, (arylazo)oxyl radical based systems, substituted and non-substituted triphenyls, verdazyl, triazolinyl, selenyl based systems (see T. S. Kwon, S. Kumazawa, T. Yokoi, S. Kondo, H. Kunisada, Y. Yuki, J. Macromol. Sci., Pure Appl. Chem. 1997, A34, 1553), tetraphenylethane derivatives, and linkers mentioned in Kamigaito et al and Hawker et al, both cited above.

Linkers that are specifically suitable for ROP are compounds that comprise an OH—, NH₂— or Tosylate (OTs) group, the latter being an initiator for ROP of oxazolines.

For the avoidance of doubt it is indicated that the linker molecule X and Y may be present at two positions within copolymer of the present invention, which is between the core (A) and the hydrophobic block (B) and/or between the hydrophobic block (B) and the hydrophilic block (D). Of course, if two linkers are present at both positions (X and Y), the linkers may have the same or different structures, independent of each other. The linkers of the copolymer of the present invention may thus be selected independently of each other within the above-given lists as is convenient to the skilled person.

The linker moiety (X and/or Y) may be a compound that is composed of several of the above-mentioned compounds in order to create a linker that is branched, resulting in t and/or z>1, for example.

In an embodiment, the copolymer according to the present invention further comprises at least one lipophilic functional agent encapsulated in or associated to the copolymer. In a preferred embodiment of the present invention, the functional agent is selected from the group of a flavour, a fragrance, a drug, an agrochemical, a dye, and mixtures thereof. The term “functional molecule” or “functional agent” refers to a molecule, which has a specific, desired activity or function. Accordingly, a functional agent may be a drug, such as a medicament for humans or animals, vitamins, trace elements, for example. It may be an agrochemical, which includes herbicides, pesticides, fungicides, and the like.

Further examples of functional agents are food additives, such as fats, oils, acidulants, dough conditioners, meat processing aids, colorants, leavening agent, minerals and enzymes. Functional agents may thus be any agent that provides a certain benefit, for example a nutritive or health benefit, to a product, for example a food or perfumed product.

Preferably, the functional agent is pharmaceutical agent. Preferably, it has a biological activity.

Alternatively, the functional agent can be a flavour and/or a fragrance. By the term “flavour” is meant a compound, which is used alone or in combination with other compounds, to impart a desired gustative effect. To be considered as a flavour, it must be recognised by a skilled person in the art as being able to modify in a desired way the taste of a composition. Such compositions are intended for oral consumption and are hence often foods, nutritional compositions and the like. Respectively, the term “fragrance” refers to a compound, which is used alone or in combination with other compounds, to impart a desired olfactive effect. To be considered as a fragrance, it must be recognised by a skilled person in the art as being able to modify in a desired way the odour of a composition.

The textbook Steffen Arctander “Perfume and Flavour Chemicals”, published by the author, 1969, is a collection of perfumes and flavours known to the skilled person and is expressly incorporated herein in its entirety by reference. Similarly, “Fenaroli's Handbook of Flavour Ingredients”, CRC Press or Synthetic Food Adjuncts by M. B. Jacobs, van Nostrand Co., Inc. are collections of flavours and/or fragrances well known to the skilled person in the art of perfuming, flavouring and/or aromatising consumer products, i.e. of imparting an odour or taste to a consumer product. The compounds disclosed in these references are flavours and/or fragrances in the context of the present invention.

In an embodiment, the copolymer of the present invention is a multiple-arm star block copolymer. Multiple-arm star block copolymers are copolymers in which a multitude of polymerised arms extend from a central structure, which gives the polymer a star-shaped appearance, and depending on the number of arms, may provide an overall spherical capsule. In addition, the arms comprise different blocks of polymers, whereby each block may be polymers from chemically similar or totally different monomeric moieties. The arms of the star block copolymer may be linear and/or branched.

In an aspect of the present invention, a process for manufacturing a block copolymer is provided. Accordingly, a core (A) is provided, which is defined as A above and which is commercially available or the synthesis of which has been discussed in the literature.

In an optional step of the process, the functionalities of the core (A) are attached to a linker moiety (X). Depending on the selected structures of A and X, the skilled person may select suitable reaction conditions, such as an adequate solvent for this reaction.

In a further step, a hydrophobic block (B) is polymerised onto the functionality of the core (A) or, if present, onto the linker moiety. Monomeric moieties for the hydrophobic block B are discussed above. Depending on the specific polymerisation reaction selected, the skilled person will adjust the reaction conditions. Preferably, the polymerisation of block B is carried out at a temperature between 20-150° C. in the presence of a catalyst.

In a further, optional step, a possible further linker moiety (Y) is connected onto the hydrophobic block (B). Of course, this linker may be selected independently from the optionally present linker following the core (A), and may thus have the same or a different structure.

In a further step of the process, a hydrophilic block (D) is polymerised directly onto the functionality of the hydrophobic block (B) or onto the optional further linker moiety (Y), or, alternatively, a further hydrophobic block is polymerised onto block (B) or onto the optional linker (Y), if present, followed by a transformation of this hydrophobic block into a hydrophilic block (D) by chemical modification, for example hydrolysis of a hydrophobic residue.

Similarly to what is said for block B above, the reaction conditions largely depend on the type of polymerisation employed and can be determined accordingly by the skilled person, who knows the optimal conditions for various polymerisation reactions.

The present invention also encompasses compounds according to formula (I), in which the positions of the hydrophobic block (B) and the hydrophilic block (D) are inversed, the other components of the compound (A, X, Y), remaining unchanged. These compounds are preferred, for example, if a hydrophilic functional agent is to be encapsulated, and the nanocapsules are dispersed or dissolved in a hydrophobic matrix, for example in an unguent, or in the oily phase of an emulsion.

Thus, the present invention also encompasses compounds of the general formula A-X-D-Y—B, with the meaning of the components remaining as discussed above. Similarly, the synthesis as well as the monomeric moieties of such compounds remain the same as disclosed above, with the exception the block D is polymerised instead of block B and block B instead of block D, respectively.

The present invention provides a perfumed product comprising the block copolymer of the invention. Examples of perfumed products include fine fragrances (perfumes, eau de toilette), body care products such as shampoos, other hair care products, shower gels, body lotions, creams, after shaves, shaving creams, soaps, home care products, such as laundry products washing agents, fabric softeners, liquid detergents, and so forth.

Preferably, the perfumed product is a perfume formulation. These kinds of products are well defined in US patent application 2003/0148901, in particular paragraphs [0026-0034], which are specifically incorporated herein by reference.

In particularly preferred embodiments, the perfumed product is fine fragrance. Preferably, these are solutions of perfuming ingredients in alcohol, emulsions or other solvents and/or carrier systems. In these applications, the slow release effect of the copolymer of the invention becomes particularly useful and convenient. For example, when the perfume including the copolymer is applied to a surface (textiles, skin, etc), for example by spraying and/or dispersing, the fragrance or perfuming ingredients will slowly be released from the surface resulting in a longer-lasting perfuming effect.

The invention will be now described in a more detailed manner by way of examples in which temperatures are indicated in degrees Celsius, and the abbreviations have the usual meaning in the art. These examples represent typical ways of carrying out the invention and should not be interpreted restrictively, in particular as regards the relative or absolute proportions of the ingredients mentioned.

### EXAMPLES

The following examples are further illustrative of the embodiments of the invention, and demonstrate the advantages of the invention relative to the prior art teachings. The NMR spectral data were recorded at 400 or 500 MHz for ¹H and at 101 or 126 MHz for ¹³C, the chemical displacement δ is indicated in ppm with respect to TMS as standard, ¹H NMR integrations represent the number of hydrogens located on one branch of the polymer, and all the abbreviations have the usual meaning in the art. UV/Vis spectra were recorded on a Perkin Elmer Lambda 900 instrument. The thermogravimetry analyser used was a Mettler Toledo Module TGA/SDTA 851e.

Commercially available reagents and solvents were used without further purification if not stated otherwise. Reactions were carried out in standard glassware under inert atmosphere. GPC analyses were carried out on a Waters 150cv instrument (modified for differential viscometry) equipped with two consecutive TSK-Gel Alpha 3,000+4,000 and/or 4,000+5,000 columns and eluted at 60° C. with dimethylformamide (DMF) containing 1 g/L of LiBr at a flow rate of 0.6 mL/min. Poly(methyl methacrylates) (PMMA) of known molecular weight were used as calibration standards. The polymer concentration was 4 mg/mL.

The examples illustrate, without limiting the scope of the invention, details for exemplified process steps set fourth above, while also showing optimal reaction conditions for different polymerisation types.

### Example 1

Preparation of Amphiphilic Star Block Copolymers by a Two Step Ring-Opening and Atom Transfer Radical Polymerisation Process

A Boltorn® H40 HBP (origin: Perstorp, Sweden) was used as initiator for the ring-opening polymerisation of ε-caprolactone. Along with the Boltorn® H40 HBP itself, the resulting poly(ε-caprolactone) (PCL) blocks provide the lipophilic interior of the final many-arm star block copolymer. In order to graft hydrophilic blocks to the precursor, functional groups serving as initiators for ATRP, such as 2-bromoisobutyryl bromide, were introduced to the ends of the PCL arms (linker moiety Y). Subsequent polymerisation of monomers such as polyethylene glycol methacrylate (PEGMA), or tert-butyl acrylate (tert-BuA) give poly(polyethylene glycol methacrylate) (PPEGMA) and poly(tert-butyl acrylate) (Ptert-BuA), respectively. In the former case the outer shell is sufficiently hydrophilic to be dispersed in water, in the latter case removal of the tert-butyl ester protective groups afforded the corresponding poly(acrylic acid) (PAA) rendering the molecule water-soluble.

### 1.i Preparation of Block Bₚ on a Commercially Available Hyperbranched Core: Polymerisation of ε-Caprolactone onto Boltorn® H40 HBP to Obtain H40-(PCL)ₚ

### Synthesis of H40-(PCL)₁₇

After precipitation into diethyl ether, Boltorn®H40 HBP (Mₙ˜7300 g/mol) was dried under vacuum for 2 days. ε-Caprolactone was dried over CaH₂ and distilled before use. A 250 mL three-neck flask was charged with Boltorn® H40 HBP (2.50 g, 5.65·10⁻⁴ mol) under an inert atmosphere and placed in an oil bath at 107° C. ε-Caprolactone (43 mL, 407 mmol) was slowly introduced. A catalytic amount of tin 2-ethylhexanoate was added. The polymerisation reaction mixture was stirred for 21 h, diluted with THF (100 mL), and precipitated into cold heptane (800 mL) to give 45.5 g (93%) of a white crystalline powder.

¹H-NMR (500 MHz, CDCl₃): 4.05 (t, 32 H); 3.65 (t, 2 H); 2.31 (t, 34 H); 1.70-1.60 (m, 68H); 1.45-1.32 (m, 34 H).

¹³C-NMR (126 MHz, CDCl₃): 173.55 (s); 64.16 (t); 34.13 (t); 28.37 (t); 25.55 (t); 24.59 (t).

GPC (DMF): Mₙ˜90000 g/mol, M_{w}/Mₙ=1.99.

A degree of polymerisation DPₚ=17 corresponding to the number of repeated units of caprolactone per arm was determined from ¹H-NMR spectroscopy according to the following equation:

$${DP}_{p} = \frac{{I}_{{CH}_{2} OCO}}{{I}_{{CH}_{2} OH}}$$

with
I_{CH2OH} corresponding to the integral of the methylene group at the end of each branch and I_{CH2OCO} corresponding to the integral of the methylene groups next to the ester function. The average structure of the compound was therefore assigned as H40-(PCL)₁₇.

### Synthesis of H40-(PCL)₁₀

As described above with 2.00 g of Boltorn® H40 HBP and 17.4 mL of ε-caprolactone for 16 h to give 19.6 g (95%) of a white crystalline powder.

¹H-NMR (500 MHz, CDCl₃): 4.05 (t, 18 H); 3.65 (t, 2 H); 2.31 (t, 20 H); 1.70-1.60 (m, 40 H); 1.45-1.32 (m, 20 H).

¹³C-NMR (126 MHz, CDCl₃): 173.55 (s); 64.16 (t); 34.13 (t); 28.37 (t); 25.55 (t); 24.59 (t).

GPC (DMF): Mₙ˜65380 g/mol, M_{w}/Mₙ=2.03.

A degree of polymerisation DPₚ=10 corresponding to the number of repeated units of caprolactone per arm was determined, and the average structure of the compound was therefore assigned as H40-(PCL)₁₀.

### Synthesis of H40-(PCL)₅₀

As described above with 0.40 g of Boltorn® H40 HBP and 19.09 mL of ε-caprolactone for 14 h to give 19.8 g (94.6%) of a white crystalline powder.

¹H-NMR (500 MHz, CDCl₃): 4.05 (t, 98 H); 3.65 (t, 2 H); 2.31 (t, 100 H); 1.70-1.60 (m, 200 H); 1.45-1.32 (m, 100 H).

¹³C-NMR (126 MHz, CDCl₃): 173.55 (s); 64.16 (t); 34.13 (t); 28.37 (t); 25.55 (t); 24.59 (t).

GPC (DMF): Mₙ˜155590 g/mol, M_{w},/Mₙ=2.06.

A degree of polymerisation DPₚ=50 corresponding to the number of repeated units of caprolactone per arm was determined, and the average structure of the compound was therefore assigned as H40-(PCL)₅₀.

Using similar conditions the following compounds were prepared and fully characterised: H40-(PCL)₂₀, H40-(PCL)₂₈ and H40-(PCL)₄₀.

### 1.ii Grafting of a Linker Moiety Y onto Block B: The Functionalisation of PCL to Obtain H40-(PCL)ₚ-Y

### Synthesis of H40-(PCL)₁₇-Y

H40-(PCL)₁₇ (43 g, 5.79·10⁻⁴ mol) were dried under vacuum for 15 minutes. Dried THF (108 mL) was added, followed by 2-bromo isobutyryl bromide (5.2 mL, 4.17·10⁻² mol), introduced dropwise from a syringe, and finally triethylamine (5.8 mL, 4.17·10⁻² mol). The reaction was carried out at ambient temperature and terminated after 65 h. The reaction mixture was precipitated into cold water and after drying under vacuum for 2 h, the polymer was again precipitated into cold water and then into heptane. After drying for one night under vacuum at 50 C., 43.3 g (93%) of H40-(PCL)₁₇-Y were obtained as a white crystalline powder.

¹H-NMR (500 MHz, CDCl₃): 4.17 (t, 2 H); 4.05 (t, 32 H); 2.31 (t, 34 H); 1.93 (s, 6 H); 1.70-1.57 (m, 68 H); 1.43-1.33 (m, 34 H).

¹³C-NMR (126 MHz, CDCl₃): 173.54 (s); 171.68 (s); 64.15 (t); 55.93 (s); 34.13 (t); 30.77 (q); 28.07 (t); 25.54 (t); 24.59 (t).

GPC (DMF): Mₙ˜106000 g/mol, M_{w}/Mₙ=1.79.

Conversion: 100%.

### Synthesis of H40-(PCL)₁₀-Y

As described above with 15 g of H40-(PCL)₁₀, 2.95 mL of 2-bromo isobutyryl bromide (2.37 10⁻² mol) and 3.30 mL of triethylamine (2.37 10⁻² mol) for 48 h to give 9.50 g (56.6%) of H40-(PCL)₁₀-Y as a white crystalline powder.

¹H-NMR (500 MHz, CDCl₃): 4.17 (t, 2 H); 4.05 (t, 18 H); 2.31 (t, 20 H); 1.93 (s, 6 H); 1.70-1.57 (m, 40 H); 1.43-1.33 (m, 20 H).

¹³C-NMR (126 MHz, CDCl₃): 173.54 (s); 171.68 (s); 64.15 (t); 55.93 (s); 34.13 (t); 30.77 (q); 28.07 (t); 25.54 (t); 24.59 (t).

GPC (DMF): Mₙ˜54350 g/mol, M_{w}/Mₙ=2.27.

### Synthesis of H40-(PCL)₅-Y

As described above with 15 g of H40-(PCL)₅₀, 3.13 mL of 2-bromo isobutyryl bromide (2.53·10⁻² mol) and 2.10 mL of triethylamine (1.51 ·10⁻² mol) for 63 h to give 14.10 g (93.4%) of H40-(PCL)₅₀-Y as a white crystalline powder.

¹H-NMR (500 MHz, CDCl₃): 4.17 (t, 2 H); 4.05 (t, 98 H); 2.31 (t, 100 H); 1.93 (s, 6 H); 1.70-1.57 (m, 200 H); 1.43-1.33 (m, 100 H).

¹³C-NMR (126 MHz, CDCl₃): 173.54 (s); 171.68 (s); 64.15 (t); 55.93 (s); 34.13 (t); 30.77 (q); 28.07 (t); 25.54 (t); 24.59 (t).

GPC (DMF): Mₙ˜125700 g/mol, M_{w}/Mₙ=2.38.

Using similar conditions the following compounds were prepared and fully characterised: H40-(PCL)₂₀-Y, H40-(PCL)₂₈-Y and H40-(PCL)₄₀-Y.

### 1.iii Polymerisation of Block D Onto the Linker Moiety Y: ATRP of tert-BuA from H40-(PCL)ₚ-Y to obtain H40-(PCL)ₚ-Y-(Ptert-BuA)_{q}

### Synthesis of H40-(PCL)₁₇Y-(Ptert-BuA)₅₀

A three-neck flask was charged with the multifunctional macroinitiator (H40-(PCL)₁₇-Y) (7 g, 8.758·10⁻⁵ mol), ethylene carbonate (4.04 g, 10% wt.) and 2,2′-bipyridyl (984.80 mg, 6.306·10⁻³ mol) and the contents dried under vacuum for 1 h 30. tert-BuA (40.41 g, 45.76 mL, 0.315 mol) was added after purification (to remove any inhibitor) and the resulting mixture was subjected to three freeze-vacuum-thaw cycles. Addition of CuBr (452 mg, 3.153·10⁻³ mol) was followed by one further freeze-vacuum-thaw cycle. The flask was then placed in a thermostatically controlled oil bath at 100° C. After 17 h, the reaction was terminated by placing the flask in an ice bath. After stirring, the polymer was diluted in THF and the contents were passed through a column of neutral alumina to remove copper salts. The THF was evaporated and the polymer precipitated into a mixture of methanol/water 9:1 (v/v), filtered and dried under vacuum for several hours. The degree of conversion was determined by NMR and confirmed with GPC.

¹H-NMR (500 MHz, CDCl₃): 4.06 (t, 32 H); 2.33-2.15 (m, br., 50 H); 2.31 (t, 34 H); 1.70-1.60 (m, 68 H); 1.58-1.30 (m, 484 H).

¹³C-NMR (126 MHz, CDCl₃): 174.20 and 173.97 (s); 173.54 (s); 80.34 (s); 64.15 (t); 42.37 and 41.9 (d); 34.13 (t); 30.33 (q); 28.03 (q); 28.37 (t); 25.55 (t); 24.59 (t).

GPC (DMF): Mₙ˜263500 g/mol, M_{w}/Mₙ=2.27.

Conversion: 50%.

A degree of polymerisation DP_{q}=50 corresponding to the number of repeated units of tert-BuA per arm was determined from ¹H-NMR spectroscopy according to the following equation:

$${DP}_{q} = \frac{{I}_{( m , br ; 1.60 - 1.28 ppm )} - {I}_{{CH}_{2 ( PCL )}}}{9 * \frac{{I}_{{CH}_{2 ( PCL )}}}{2 * {DP}_{P}}}$$

with
DPₚ being the polymerisation degree for the polycaprolactone,
I_{(m,br;1.60-1.28 ppm)}-I_{CH}_{_{2(PCL)}} corresponding to the integral of the tert-butyl group of tert-BuA and I_{CH}_{_{2(PCL)}} corresponding to the integral of the methylene group of PCL at 4.06 ppm.
   The average structure of the compound was therefore assigned as H40-(PCL)₁₇-Y-(Ptert-BuA)₅₀.

### Synthesis of H40-(PCL)₁₀-Y-(Ptert-BuA)₇₀

As described above with 1 g of H40-(PCL)₁₀-Y, 2.05 g of ethylene carbonate, 222 mg (1.41·10⁻³ mol) of 2,2′-bipyridyl, 20.6 mL of tert-BuA (1.41 10⁻¹ mol) and 101.6 mg (7.08·10⁻4 mol) of CuBr for 21 h at 90° C. to give 14.28 g of a white crystalline powder.

¹H-NMR (500 MHz, CDCl₃): 4.06 (t, 18 H); 2.40-2.10 (m, 70 H); 2.30 (t, 20 H); 1.70-1.60 (m, 40 H); 1.59-1.30 (m, 650 H).

¹³C-NMR (126 MHz, CDCl₃): 174.20 and 173.97 (s); 173.54 (s); 80.34 (s); 64.15 (t); 42.37 and 41.9 (d); 34.13 (t); 30.33 (q); 28.03 (q); 28.37 (t); 25.55 (t); 24.59 (t).

GPC (DMF): Mₙ˜459740 g/mol, M_{w}/Mₙ=1.84.

A degree of polymerisation DP_{q}=70 corresponding to the number of repeated units of tert-BuA per arm was determined, and the average structure of the compound was therefore assigned as H40-(PCL)₁₀-Y-(Ptert-BuA)₇₀.

### Synthesis of H40-(PCL)₁₀-Y-(Ptert-BuA)₁₁₅

As described above with 1 g of H40-(PCL)₁₀-Y, 2.05 g of ethylene carbonate, 222 mg (1.41·10⁻³ mol) of 2,2′-bipyridyl, 20.6 mL of tert-BuA (1.41·10⁻¹ mol) and 101.6 mg (7.08·10⁻4 mol) of CuBr for 48 h at 90° C. to give 14.28 g of a white crystalline powder.

¹H-NMR (500 MHz, CDCl₃): 4.06 (t, 18 H); 2.34-2.15 (m, 115 H); 2.30 (t, 20 H); 1.70-1.61 (m, 40 H); 1.60-1.28 (m, 1055 H).

¹³C-NMR (126 MHz, CDCl₃): 174.20 and 173.97 (s); 173.54 (s); 80.34 (s); 64.15 (t); 42.37 and 41.9 (d); 34.13 (t); 30.33 (q); 28.03 (q); 28.37 (t); 25.55 (t); 24.59 (t).

GPC (DMF): Mₙ˜549000 g/mol, M_{w}/Mₙ=2.06.

A degree of polymerisation DP_{q}=115 corresponding to the number of repeated units of tert-BuA per arm was determined, and the average structure of the compound was therefore assigned as H40-(PCL)₁₀-Y-(Ptert-BuA)₁₁₅.

### Synthesis of H40-(PCL)₅₀-Y-(Ptert-BuA)₅₄

As described above with 2 g of H40-(PCL)₅₀-y, 972 mg of ethylene carbonate, 104.6 mg (6.7 10⁻⁴ mol) of 2,2′-bipyridyl, 9.72 mL of tert-BuA (6.69·10⁻² mol) and 48 mg (3.3·10⁻⁴mol) of CuBr for 20 h at 90° C. to give 3.31 g of a white crystalline powder.

¹H-NMR (500 MHz, CDCl₃): 4.06 (t, 98 H); 2.30-2.15 (m, 54 H); 2.30 (t, 100 H); 1.75-1.60 (m, 200 H); 1.59-1.28 (m, 586 H).

¹³C-NMR (126 MHz, CDCl₃): 174.20 and 173.97 (s); 173.54 (s); 80.34 (s); 64.15 (t); 42.37 and 41.9 (d); 34.13 (t); 30.33 (q); 28.03 (q); 28.37 (t); 25.55 (t); 24.59 (t).

GPC (DMF): Mₙ˜376280 g/mol, M_{w}/Mₙ=2.27.

A degree of polymerisation DP_{q}=54 corresponding to the number of repeated units of tert-BuA per arm was determined, and the average structure of the compound was therefore assigned as H40-(PCL)₅₀-Y-(Ptert-BuA)₅₄.

Using similar conditions the following compounds were prepared and fully characterised: H40-(PCL)₁₀-Y-(Ptert-BuA)₁₇, H40-(PCL)₁₀-Y-(Ptert-BuA)₆₈, H40-(PCL)₁₇-Y-(Ptert-BuA)₁₈, H40-(PCL)₁₇-Y-(Ptert-BuA)₂₀, H40-(PCL)₅₀-Y-(Ptert-BuA)₂₂, H40-(PCL)₅₀-Y-(Ptert-BuA)₂₈ and H40-(PCL)₅₀-Y-(Ptert-BuA)₆₄.

### 1.iv Further Modification of Block D to Obtain the Desired Value of the Hansen Solubility Parameter: Hydrolysis of the Tert-butyl Groups from H40-(PCL)ₚ-Y-(Ptert-BuA)_{q} to Obtain H40-(PCL)ₚ-Y-(PAA)_{q}

### Synthesis of H40-(PCL)₁₇-Y-(PAA)₅₀

Multifunctional star polymer H40-(PCL)₁₇-Y-(Ptert-BuA)₅₀ (10 g, 3.253·10⁻⁵ mol) was dissolved in dichloromethane (100 mL). Then trifluoroacetic acid (43 mL, 5.854·10⁻¹ mol) was added to the flask. The solution was stirred for 2 h at room temperature before the solvent was removed by evaporation. The product was redissolved in THF (60 mL), precipitated into 650 mL heptane and dried under vacuum for 3 d at 50° C. to give 4.98 g of H40-(PCL)₁₇-Y-(PAA)₅₀ as a white powder of the partially hydrolysed product (to more than 30%).

¹H-NMR (500 MHz, DMSO-d6): 12.23 (s, br.); 3.99 (t); 2.32-2.14 (m); 1.85-1.67 (m); 1.63-1.22 (m).

¹³C-NMR (125.8 MHz, DMSO-d6): 175.78 (s); 175.63 (s); 172.67 (s); 63.39 (t); 41.70-40.50 (d, br.); 36.50-34.00 (t, br.); 33.27 (t); 27.69 (t); 23.98 (t).

GPC (water): Mₙ˜46800 g/mol, M_{w}/Mₙ=1.26.

### Synthesis of H40-(PCL)₁₀-Y-(PAA)₇₀

As described above with 3.22 g (8·10⁻⁶ mol) of H40-(PCL)₁₀-Y-(Ptert-BuA)₇₀ in 36.5 mL of dichloromethane and 16.5 mL (2.22·10⁻¹ mol) of trifluoroacetic acid. The product was redissolved in ethanol (30 mL), precipitated into 300 mL of ether and dried under vacuum for 3 d to give 1.75 g of H40-(PCL)₁₀-Y-(PAA)₇₀ as a white powder of the partially hydrolysed product (to more than 30%).

¹H-NMR (400 MHz, DMSO-d6): 12.35 (s, br.); 4.10-3.95 (m); 2.42-2.17 (m); 1.95-1.75 (m); 1.72-1.20 (m).

¹³C-NMR (101 MHz, DMSO-d6): 175.78 (s); 175.63 (s); 172.67 (s); 63.39 (t); 41.70-40.50 (d, br.); 36.50-34.00 (t, br.); 33.27 (t); 27.69 (t); 23.98 (t).

GPC (water): Mₙ˜381800 g/mol, M_{w}/Mₙ=2.79.

### Synthesis of H40-(PCL)₁₀-Y-(PAA)₁₁₅

As described above with 4.56 g (7.9·10⁻⁶ mol) of H40-(PCL)₁₀-Y-(Ptert-BuA)₁₁₅ in 52 mL of dichloromethane and 24.3 mL (3.27·10⁻¹ mol) of trifluoroacetic acid for 2 h 15. The product was redissolved in ethanol (30 mL), precipitated into 300 mL of ether and dried under vacuum for 2 d to give 2.28 g of H40-(PCL)₁₀-Y-(PAA)₁₁₅ as a white powder of the partially hydrolysed product (to more than 30%).

¹H-NMR (400 MHz, DMSO-d6): 12.31 (s, br.); 4.20-3.90 (m); 2.52-2.10 (m); 2.05-1.74 (m); 1.72-1.20 (m).

¹³C-NMR (101 MHz, DMSO-d6): 175.78 (s); 175.63 (s); 172.67 (s); 63.39 (t); 41.70-40.50 (d, br.); 36.50-34.00 (t, br.); 33.27 (t); 27.69 (t); 23.98 (t).

GPC (water): Mₙ˜312400 g/mol, M_{w}/Mₙ=2.31.

### Synthesis of H40-(PCL)₅₀-Y-(PAA)₅₄

As described above with 0.79 g (1.82·10⁻⁶ mol) of H40-(PCL)₅₀-Y-(Ptert-BuA)₅₄ in 11.5 mL of dichloromethane and 1.95 mL (2.63·10⁻² mol) of trifluoroacetic acid for 1 h. The product was redissolved in ethanol (20 mL), precipitated into 200 mL of ether and dried under vacuum overnight to give 333.5 mg of H40-(PCL)₅₀-Y-(PAA)₅₄ as a white powder of the partially hydrolysed product (to at least 27%).

¹H-NMR (400 MHz, DMSO-d6): 12.34 (s, br.); 4.35-3.79 (m); 2.45-2.14 (m); 1.95-1.73 (m, br.); 1.72-1.22 (m).

¹³C-NMR (101 MHz, DMSO-d6): 175.78 (s); 175.63 (s); 172.67 (s); 63.39 (t); 41.70-40.50 (d, br.); 36.50-34.00 (t); 33.27 (t); 27.69 (t); 23.98 (t).

GPC (DMF): Mₙ˜541160 g/mol, M_{w}/Mₙ=4.65.

### 1.v Polymerisation of Hydrophilic Block D onto the Linker Moiety Y: ATRP of PEGMA from H40-(PCL)ₚ-Y to Obtain H40-(PCL)ₚ-Y-(PPEGMA)_{q}

A three-neck flask was charged with the multifunctional macroinitiator (H40-(PCL)₁₇-Y) (0.5 g, 6.256·10⁻⁶ mol), ethylene carbonate (990 mg, 10% wt.) and 2,2′-bipyridyl (70.2 mg, 4.5·10⁻⁴ mol) and the contents dried under vacuum for 1 h. Purified PEGMA (10.70 g, 9.9 mL, 2.25·10⁻² mol, with 8 ethylene glycol units) and 5 mL of distilled toluene were added; the resulting mixture was subjected to three freeze-vacuum-thaw cycles. Addition of CuBr (32 mg, 2.25·10⁻⁴ mol) was followed by one further freeze-vacuum-thaw cycle. The flask was then placed in a thermostatically controlled oil bath at 75° C. After 4 h 45, the reaction was terminated by placing the flask in an ice bath. After stirring, the toluene was evaporated and the polymer was dispersed in water. The white dispersion was placed in a dialysis bag with molecular weight cut off of 6-8,000 g/mol. After three days, the dispersion was lyophilised and 1.16 g (10.9%) of a white, weak and sticky powder were obtained.

¹H-NMR (500 MHz, DMSO-d6): 3.97 (s, br., 32 H); 3.51 (s, 192 H); 3.24 (s, 18 H); 2.26 (t, br, 34 H); 1.53 (m, br., 68 H); 1.30 (t, br., 34 H).

¹³C-NMR (126 MHz, CDCl₃): 172.53 (s); 69.72 (t, 16x); 63.34 (t); 57.94 (s); 33.24 (t); 27.71 (t); 24.8 (t); 23.98 (t).

A degree of polymerisation DP_{q}=6 corresponding to the number of repeated unit of PEGMA per arm was determined from ¹H-NMR spectroscopy according to the following equation:

$${DP}_{q} = \frac{{I}_{{CH}_{3 ( PEGMA )}}}{3 * \frac{{I}_{{CH}_{2 ( PCL )}}}{2 * {DP}_{P}}}$$

with
I_{CH}_{_{3(PEGMA)}} corresponding to the integral of the terminal methyl group of PEGMA at 3.24 ppm
   and
I_{CH}_{_{2(PCL)}} corresponding to the integral of the methylene group of PCL at 3.97 ppm.
   The average structure of the compound was therefore assigned as H40-(PCL)₁₇-Y-(PPEGMA)₆.

### Example 2

### Preparation of an Amphiphilic Star Block Copolymer by a Two Step Radical Atom Transfer Polymerisation Process

A Boltorn® H40 HBP was used to introduce functional groups capable of initiating ATRP directly onto the core. The lipophilic block B was built up via an initial ATRP step using a monomer such as methyl methacrylate (MMA) to give poly(methyl methacrylate) (PMMA) or n-butyl methacrylate (n-BuMA) to give poly(n-butyl methacrylate) (Pn-BuMA), respectively. A second ATRP step was used as in Example 1 to create the hydrophilic block D. For example, polymerisation of polyethylene glycol methacrylate (PEGMA) gives poly(polyethylene glycol methacrylate) (PPEGMA).

### 2.i Grafting of the Linker Moiety X onto the Hyperbranched Polymer A to Obtain H40-X

A solution of vacuum-dried Boltorn® H40 HBP (2.8 g) in 80 mL of dry THF, containing a total of 25 mmol of hydroxyl functions, was added to a solution of 4-(dimethylamino)pyridine (4.79 g, 39.3 mmol) and triethylamine (2.53 g, 3.48 mL, 25.0 mmol) in dry THF (20 mL) under an inert atmosphere. Then, 2-bromoisobutyric acid bromide (17.24 g, 9.27 mL, 75.0 mmol) was added dropwise at room temperature. After 48 h, precipitated salts were filtered off and the solvent partially evaporated. The residual solution was poured into methanol and the precipitate dried under vacuum.

¹H-NMR (400 MHz, CDCl₃): 4.40-4.22 (m, 144 H); 1.89 (s, 216 H); 1.25-1.33 (m, 108 H).

¹³C-NMR (101 MHz, CDCl₃): 171.6 (s); 171.4 (s); 170.8 (s); 66.0 (m); 55.4 (s); 46.7 (s); 30.6 (q); 17.8 (q).

GPC (DMF): Mₙ˜12300 g/mol, M_{w}/Mₙ=1.72.

### 2.ii Polymerisation of Lipophilic Block B by ATRP Starting from the Linker Moiety X to Obtain H40-X-(PMMA)ₚ

A flask equipped with a nitrogen inlet was charged with the macroinitiator H40-X (132.5 mg, ca. 0.5 mol of initiating groups), toluene (25 g), freshly distilled MMA (25 g, 250 mmol), CuBr (140 mg, 1.0 mmol) and n-propyl-2-pyridinylmethaneimine (296 mg, 2.0 mmol). The mixture was subsequently deoxygenated by three freeze pump-thaw cycles. Polymerisation was carried out in a thermostatically controlled oil bath at 60° C. and the degree of conversion followed by ¹H-NMR spectroscopy of samples taken at different stages of the reaction using a nitrogen purged gas tight syringe. For work-up, the catalyst complex was removed by passing the reaction mixture through a short silica gel column. The resulting polymer solution was finally precipitated into methanol (20 times the volume of the reaction mixture).

¹H-NMR (400 MHz, CDCl₃): 4.40-4.00 (s, 3 H); 3.59 (s, 30 H); 2.20-0.70 (m, 68 H).

¹³C-NMR (101 MHz, CDCl₃): 178.0 (s); 177.7 (s); 176.9 (s); 54.4 (m); 51.8 (s); 44.8 (s); 44.8 (s); 44.5 (s); 18.9 (d); 16.3 (d).

GPC (DMF): Mₙ˜86000 g/mol, M_{w}/Mₙ=1.82.

Conversion: 33%.

A degree of polymerisation DP_{P}=10 corresponding to the number of repeated units of MMA per arm was determined from ¹H-NMR spectroscopy according to the following equation:

$${DP}_{p} = \frac{{I}_{{COOCH}_{3 ( PMMA )}}}{{I}_{{COOCH}_{3 ( PMMA )}} + {I}_{{COOCH}_{3 ( MMA )}}} * {DP}_{targeted}$$

with
I_{COOCH}_{_{3(PMMA)}} corresponding to the integral of the methyl group of PMMA at 3.59 ppm,
I_{COOCH}_{_{3(MMA)}} corresponding to the integral of the methyl group of MMA and
DP_{targeted} being the expected degree of polymerisation.
   The average structure of the compound was therefore assigned as H40-X-(PMMA)₁₀.

### 2.iii ATRP of the Hydrophilic Block D to Obtain H40-X-(PMMA)ₚ-(PPEGMA)_{q}

A flask equipped with a nitrogen inlet was charged with H40-X-(PMMA)₁₀ star polymer (1.6 g, ˜0.75 mol of initiating groups), toluene (17.8 g), PEGMA (17.8 g, 50 mmol, Mₙ˜475 g/mol, previously passed over alumina to remove inhibitors), CuBr (215 mg, 1.5 mmol) and n-propyl-2-pyridinylmethaneimine (445 mg, 3.0 mmol). The mixture was subsequently deoxygenated by three freeze pump-thaw cycles. Polymerisation was carried out in a thermostatically controlled oil bath at 60° C. and the degree of conversion followed by ¹H-NMR spectroscopy of samples taken at different stages of the reaction using a nitrogen purged gas tight syringe. For work-up, the catalyst complex was removed by passing the reaction mixture through a short silica gel column. The resulting polymer solution was finally precipitated into diethyl ether (20 times the volume of the reaction mixture). The resulting milk was centrifuged and the resulting precipitate washed several times with diethyl ether to extract any remaining monomer. Further purification was carried out by dialysis in methanol (molecular weight cut off=7,000 g/mol). The product was obtained after evaporation of the solvent and vacuum drying.

¹H-NMR (400 MHz, CDCl₃): 4.05 (s, 16 H); 3.70-3.50 (m, 336 H,); 3.35 (s, 24 H); 2.20-0.70 (m, 112 H).

¹³C-NMR (101 MHz, CDCl₃, decoupled): 71.9; 70.5; 69.1; 68.4; 63.8; 58.9; 51.7; 44.8; 44.5; 18.3.

GPC (DMF): Mₙ˜277800 g/mol, M_{w}/Mₙ=1.78.

Conversion 15%.

A degree of polymerisation DP_{q}=8 corresponding to the number of repeated units of PEGMA per arm was determined from ¹H-NMR spectroscopy according to the following equation:

$${DP}_{q} = \frac{{I}_{{COOCH}_{2} {R}_{( PPEGMA )}}}{{I}_{{COOCH}_{2} {R}_{( PPEGMA )}} + {I}_{{COOCH}_{2} {R}_{( PEGMA )}}} * {DP}_{targeted}$$

I_{COOCH}_{₂}_{R}_{_{(PPEGMA)}} corresponding to the integral of the methylene groups of PPEGMA and
I_{COOCH}_{₂}_{R}_{_{(PEGMA)}} corresponding to the integral of the methylene groups of PEGMA.
   The average structure of the compound was therefore assigned as H40-X-(PMMA)₁₀-(PPEGMA)₈.

### 2.iv Polymerisation of Lipophilic Block B by ATRP Starting from the Linker Moiety X to Obtain H40-X-(Pn-BuMA)ₚ

A flask equipped with a nitrogen inlet was charged with macroinitiator H40-X (1.325 g, ˜5 mmol of initiating groups, see Example 2.i,), toluene (71.1 g), freshly distilled n-BuMA (71.1 g, 500 mmol), CuBr (700 mg, 5.0 mmol) and n-propyl-2-pyridinylmethaneimine (1.48 g, 10.0 mmol). The mixture was subsequently deoxygenated by three freeze pump-thaw cycles. Polymerisation was carried out in a thermostatically controlled oil bath at 60° C. After 100 min the reaction mixture was cooled in an ice bath, the catalyst complex was removed by suction filtration of the reaction mixture through a layer of silica gel (ca. 3 cm). The resulting polymer solution was partially evaporated and finally precipitated into methanol (20 times the volume of the reaction mixture).

¹H-NMR (400 MHz, CDCl₃): 4.42-3.80 (m, 60 H); 2.20-0.70 (m, 720 H).

¹³C-NMR (101 MHz, CDCl₃): 177.8 (s); 177.5 (s); 176.7 (s); 64.7 (s); 54.2 (m); 45.1 (s); 44.7 (s); 19.4 (s); 18.3 (s); 16.5 (s); 13.7 (s).

GPC (DMF): Mₙ˜182000 g/mol, M_{w}/Mₙ=1.69.

Conversion: 30% (after 100 min).

A degree of polymerisation DP_{P}=30 corresponding to the number of repeated units of n-BuMA per arm was determined from ¹H-NMR spectroscopy according to the following equation:

$${DP}_{p} = \frac{{I}_{{COOCH}_{2} {R}_{( PBMA )}}}{{I}_{{COOCH}_{2} {R}_{( PBMA )}} + {I}_{{COOCH}_{2} {R}_{( BMA )}}} * {DP}_{targeted}$$

with
I_{COOCH}_{₂}_{R}_{_{(PBMA)}} corresponding to the integral of the a-methylene group of Pn-BuMA at 3.95 ppm,
I_{COOCH}_{₂}_{R}_{_{(BMA)}} corresponding to the integral of the α-methylene group of n-BuMA and
DP_{targeted} being the expected degree of polymerisation.
   The average structure of the compound was therefore assigned as H40-X-(Pn-BuMA)₃₀.

### 2.v ATRP of the Hydrophilic Block D to Obtain H40-X-(Pn-BuMA)ₚ-(PPEGMA)_{q}

A flask equipped with a nitrogen inlet was charged with CuBr (140 mg, 1.0 mmol) and PEGMA (23.8 g, 50.0 mmol, Mₙ˜475 g/mol, previously passed over alumina to remove inhibitors). After degassing by bubbling nitrogen through the mixture for 30 min, n-propyl-2-pyridinylmethaneimine (380 82 L, 369 mg, 2.5 mmol) was added while degassing was continued. A previously degassed solution of H40-X-(Pn-BuMA)₃₀ (2.23 g, ˜0.5 mmol of initiating groups) in of toluene (23.8 g) was added to the mixture, nitrogen purging was continued for 15 min. The reaction flask was then put into a thermostatically controlled oil bath at 60° C. Polymerisation was stopped after 5 h by cooling the reaction mixture to 0° C. and removing the catalyst by suction filtration through a layer of silica gel (˜3 cm). Toluene was evaporated from the resulting polymer solution; the polymer was isolated and purified by repeated precipitation into diethyl ether (20 times the volume of the reaction mixture). Further purification was carried out by dialysis in water (molecular weight cut off=10000 g/mol). The product was obtained after evaporation of the solvent and vacuum drying.

¹H-NMR (400 MHz, CDCl₃): 4.06 (s, br., 48 H); 3.93 (s, br, 32 H); 3.74-3.50 (m, 992 H); 3.36 (s, 96 H); 2.20-0.70 (m, 448 H).

GPC (DMF): Mₙ˜793500 g/mol, M_{w}/Mₙ=2.06.

Conversion: 32% (after 24 h).

A degree of polymerisation DP_{q}=32 corresponding to the number of repeated units of PEGMA per arm was determined, and the average structure of the compound was therefore assigned as H40-X-(Pn-BuMA)₃₀-(PPEGMA)₃₂.

### 2.vi ATRP of the Hydrophilic Block D to Obtain H40-X-(PPEGMA)_{q} as Reference Compound Without Hydrophobic Inner Layer

As described above (Example 2.v) with 59.5 g (125.0 mmol) of PEGMA and 189 mg of H40-X (instead of H40-X-(Pn-BuMA)q) in 59.8 g of toluene at 50° C. for 18 h.

¹H-NMR (400 MHz, CDCl₃): 4.07 (s, br., 48 H); 3.83-3.43 (m, 1200 H); 3.37 (s, 120 H); 2.26-0.60 (m, 200 H).

GPC (DMF): Mₙ˜779700 g/mol, M_{w}/Mₙ=1.82.

Conversion: 16% (after 18 h).

A degree of polymerisation DP_{q}=40 corresponding to the number of repeated units of PEGMA per arm was determined, and the average structure of the compound was therefore assigned as H40-X-(PPEGMA)₄₀.

### Example 3

Encapsulation of a Lipophilic Dye Followed by UV Spectroscopy

The star block copolymer H40-X-(PMMA)₁₀-(PPEGMA)₈ (prepared as described in Example 2.iii) was used to encapsulate rubrene as a hydrophobic agent.

5 mg of the lipophilic, water insoluble dye rubrene (origin: Sigma-Aldrich) were stirred with 50 mg of the amphiphilic star block copolymer. Then, 2.0 mL of water was added to the resulting mixture and stirring was continued until the polymer was dissolved. The remaining undissolved rubrene was removed by centrifugation. The reddish supernatant was investigated by UV/Vis spectroscopy, confirming the presence of rubrene in the aqueous polymer solution. In conclusion, the copolymer of the present invention effectively encapsulates rubrene.

The evaluation of the results of the UV spectroscopy was based on the absorption maxima for rubrene in aqueous polymer solution (538 nm; 501 nm; 333 nm) as compared to the absorption maxima for rubrene in heptane solution as specified by supplier (523±3 nm; 488±3 nm; 299±3 nm).

The star block copolymer H40-X-(Pn-BuMA)₃₀-(PPEGMA)₃₂ (prepared as described in Example 2.v) was used to encapsulate Reichardt's dye as a hydrophobic agent.

For encapsulation, 28.5 mg of the amphiphilic star block copolymer and 1.7 mg of Reichardt's dye were dissolved in 4.0 ml of methanol or THF, respectively. Adding small portions of water and partial evaporating of the solvent were repeated until all organic solvent was replaced by water. The mixtures obtained were filtered through a 0.22 μm syringe filter and analysed by UV spectroscopy. The spectra were recorded on a Cary Bio 100 UV spectrometer, using quartz cuvettes with a path length of 1 cm. Solutions of Reichardt's dye in methanol and ethyl acetate were prepared as references and analysed to check the experimental setup. From the wavelength of the long wavelength absorption maxima the E_{T}(30) value characterising the polarity of the dye's environment was calculated with the formula E_{T}(30)=hcN_{A}/λₘₐₓ. The following results were obtained:

| | λₘₐₓ | | |
|---|---|---|---|
| Solvent | [nm] | E_{T}(30) exp. | E_{T}(30) lit. |
| methanol | 515 | 55.5 | 55.4 |
| ethyl acetate | 747 | 38.3 | 38.1 |

| | | |
|---|---|---|
| H40-X-(Pn-BuMA)₃₀-(PPEGMA)₃₂ in | 554 | 51.6 |
| water, encapsulation from methanol | | |
| H40-X-(Pn-BuMA)₃₀-(PPEGMA)₃₂ in | 560 | 51.1 |
| water, encapsulation from THF | | |

The results obtained for the solutions of the dye in methanol and ethyl acetate confirm the experimental setup, as the E_{T}(30) values obtained correspond well to the literature values (see: C. Reichardt, Chem Rev. 1994, 94, 2319-2358).

The E_{T}(30) values obtained for the star block copolymer containing solutions show that it is of secondary importance whether the encapsulation is performed from a non-solvent for the core (methanol) or a good solvent for the core (THF). Furthermore, it is possible to estimate the solvent properties of the system. The solubilisation of the otherwise water-insoluble dye can be explained as an encapsulating effect of the amphiphilic star block copolymer.

### Example 4

Encapsulation of a Lipophilic Dye Followed by GPC Analysis

The amphiphilic star block copolymer H40-X-(Pn-BuMA)₃₀-(PPEGMA)₃₂ (prepared as described in Example 2.v) was used to encapsulate a hydrophobic dye.

20.0 mg of the star block copolymer and 2.0 mg of rubrene were dissolved in dichloromethane and the solvent was evaporated. The resulting mixture was taken up in 4.0 ml of GPC buffer solution (0.1 mol/L aqueous NaHCO₃-solution), and filtered through a 0.22 μm syringe filter. To remove fine dye particles that passed through the filter, the mixture was extracted once with diethyl ether. The resulting solution was analysed by GPC on a Waters 150CV instrument equipped with a built-in refractive index detector, and a Kontron 430 UV detector, calibrated to 306 nm for the experiment. As separatory columns Shodex OH-pak SB-803+SB-804 (diameter 8.0 mm, length 300 mm) were used respectively. Samples were eluted with a 0.1 mol/L aqueous NaHCO₃-solution at a flow rate of 0.50 mL/min. After extraction of the free dye with diethyl ether the rubrene elutes together with the polymer at a retention volume of ca. 12 mL, thus showing the successful encapsulation of the dye into the polymer.

### Example 5

Encapsulation and Release of Fragrances Followed by UV Spectroscopy

Amphiphilic star block copolymer H40-X-(Pn-BuMA)₃₀-(PPEGMA)₃₂ (prepared as described in Example 2.v) was used to encapsulate 1-(2-naphthalenyl)-1-ethanone as a hydrophobic and UV-active fragrance molecule. The encapsulation and release properties of the polymer according to the invention were compared to that of unmodified Boltorn® H40 HBP.

10.0 mg of 1-(2-naphthalenyl)-1-ethanone and 18.7 mg of either H40-X-(Pn-BuMA)₃₀-(PPEGMA)₃₂ or Boltorn® H40 HBP, respectively, were dissolved in 4.0 ml of THF. Adding small portions of water and partial evaporation of the solvent were repeated until all organic solvent was replaced by water. A reference solution in pure water was prepared in the same manner. The mixtures obtained were filtered through a 0.22 μm syringe filter and analysed by UV spectroscopy. The spectra were recorded on a Cary Bio 100 UV spectrometer, using quartz cuvettes with a path length of 1 cm, with water as a reference. The sample containing H40-X-(Pn-BuMA)₃₀-(PPEGMA)₃₂ was diluted by 1:10 to reduce the absorbance. The measured UV absorptions of 1-(2-naphthalenyl)-1-ethanone measured at 340 nm are as follows:
in water 0.69
with Boltorn® H40 HBP in water 1.08
with H40-X-(Pn-BuMA)₃₀-(PPEGMA)₃₂ in water 1.57
   The data demonstrates the pre-eminence of H40-X-(Pn-BuMA)₃₀-(PPEGMA)₃₂ as compared to Boltorn® H40 HBP. At the same mass concentration more than ten times as much of the hydrophobic guest is encapsulated into the polymer according to the invention than into the Boltorn® H40 HBP reference.

For the comparative release experiments the cuvettes with the solutions were put at a ventilated place. After the given times the evaporated water was added and the spectra were recorded. The recorded UV absorptions at 340 nm are summarised in the table:

| | H40-X- | | H40-X- | |
|---|---|---|---|---|
| | (Pn-BuMA)₃₀- | Boltorn ® | (Pn-BuMA)₃₀- | Boltorn ® |
| Time | (PPEGMA)₃₂ | H40 HBP | (PPEGMA)₃₂ | H40 HBP |
| [h] | absorption | absorption | % retained | % retained |
| 0 | 1.57 | 1.08 | 100.0 | 100.0 |
| 36 | 1.21 | 0.36 | 77.3 | 33.3 |
| 60 | 1.03 | 0.28 | 65.6 | 26.4 |
| 150 | 0.27 | — | 16.9 | — |

The data show that the release of the fragrance molecule is slower from the amphiphilic star block copolymer H40-X-(Pn-BuMA)₃₀-(PPEGMA)₃₂ than from the reference sample. For example, after 60 h 66% of the fragrance are still in the amphiphilic star block copolymer, as compared to only 26% in the Boltorn® H40 HBP. At 150 h precipitates formed from the Boltorn® H40 HBP solution, and no trace of fragrance could be found.

### Example 6

Quantification of Fragrance Encapsulation by NMR Spectroscopy

Amphiphilic star block copolymer H40-X-(Pn-BuMA)₃₀-(PPEGMA)₃₂ (prepared as described in Example 2.v) (˜10/20/30/40 mg) was precisely weighed in and dissolved in 1.4 g of D₂O (pure D₂O was used as a blank sample). After the polymer had dissolved, 50 mg of a fragrance molecule (benzyl acetate, (E)-3,7-dimethyl-2,6-octadien-1-ol, 4-tert-butyl-1-cyclohexyl acetate (Vertenex®, origin: International Flavors & Fragrances, USA) or decanal, respectively) were added to the solutions. After shaking overnight, samples were filtered into Eppendorf caps through 0.22 μm syringe filters. After centrifugation, aliquots of the water phase were weighed into NMR tubes, and an exact amount of DMSO was added to the samples as reference for quantification. NMR spectra were recorded using the following acquisition conditions: preacquisition delay 20 s, acquisition time 5 s, number of data points 64 k, 64 scans. When processing the spectra, a line broadening of 0.1 Hz and a zero filling of 1024 k was used. Spectra were manually integrated, without additional baseline correction. The following signals were used for the quantification: benzyl acetate, —(CO)—CH₃, s, δ=2.02 to 1.81 ppm, depending on type and concentration of polymer; (E)-3,7-dimethyl-2,6-octadien-1-ol (geraniol), C═CH—C, t, δ=5.3 ppm and/or C═CH—C, t, δ=5.1 ppm; decanal, —CH₂—CHO, pert t, δ=2.1 ppm in water, 2.31 ppm in polymer solutions; Vertenex®, —C(CH₃)₃, br. s, δ=0.95 to 0.80 ppm, depending on polymer concentration. All signals are well separated from polymer signals except for Vertenex®, limiting accuracy for this probe molecule.

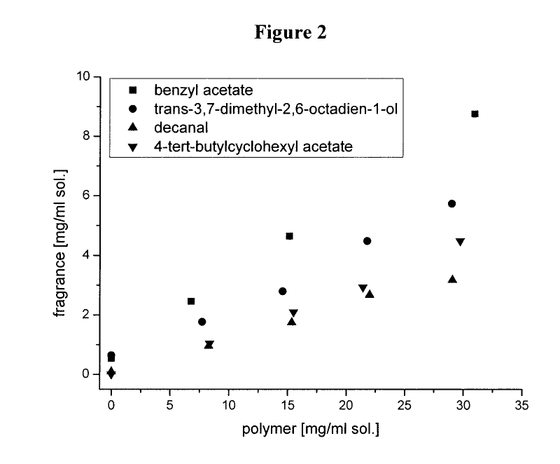

The data points given in FIG. 2 show that with an increasing amount of polymer in solution an increasing amount of fragrance is encapsulated. The data show a linear correlation between the amount of polymer in solution and the amount of quantified fragrance molecule, thus proving the successful encapsulation of the fragrance molecules in the polymer.

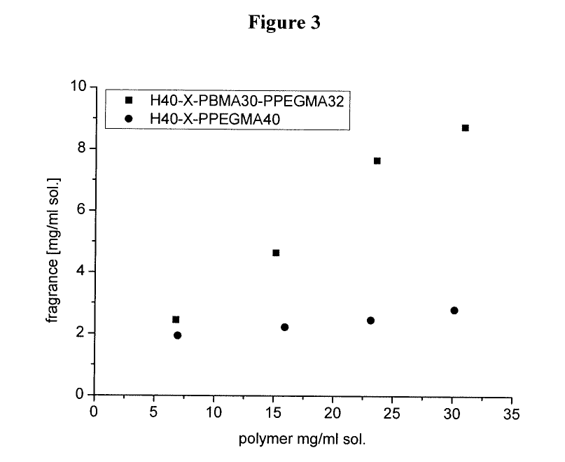

Comparing the data obtained for the encapsulation of benzyl acetate into H40-X-(Pn-BuMA)₃₀-(PPEGMA)₃₂ with those obtained for a star copolymer with no inner hydrophilic shell, such as reference compound H40-X-(PPEGMA)₄₀ (prepared in Example 2.vi), shows that a significantly higher amount of fragrance molecules can be encapsulated inside the core shell structures described in the present invention (FIG. 3), thus illustrating the advantage of having a hydrophobic block and a hydrophilic block.

### Example 7

Demonstration of Fragrance Encapsulation by Self-Diffusion NMR Spectroscopy

Samples containing fragrance molecules and amphiphilic star block copolymer were prepared as described above (Example 6). The polymer used was H40-X-(Pn-BuMA)₃₀-(PPEGMA)₃₂ in a concentration of 10.7 mg/ml in D₂O. For the measurement of fragrance molecules in pure water 1 μl of fragrance was thoroughly mixed with 700 μl D₂O. For 4-tert-butyl-1-cyclohexyl acetate (Vertenex®) and decanal further dilution was necessary.

Measurements were performed on a Bruker Avance 500 MHz NMR spectrometer equipped with a 5 mm BBQ probe and a GAD gradient amplifier capable of delivering gradient strengths up to 54 ^{G}/cm (=0.54 ^{T}/m). A double stimulated spin echo pulse sequence (see: A. Jerschow and N. Müller, J. Magn. Reson. 1997, 125, 372-375) was used to determine the diffusion coefficients in solution for both, fragrance molecules and star block copolymer. All spectra were recorded at 25° C. using a preacquisition delay of 4 s, an acquisition time of 2 s, and a spectral width of 15 ppm. The number of scans varied between 8 and 64 depending on the signal intensities of the fragrance molecules. Typically, series of 16 experiments were recorded with increasing gradient strengths (g) from 1 to 50 G/cm using trapezoidal gradient forms. The gradient duration (δ) and diffusion time (Δ) were kept constant in all experiments. Depending on the value of the apparent diffusion coefficient the values for δ (3-6 ms) and Δ (50-250 ms) were adjusted such that at 95% gradient strength the signals were damped to between 5 and 10% of the original signal intensities.

All spectra were processed using Bruker XWINNMR software. After apodisation with exponential decay functions of 1 Hz and zero-filling the data were Fourier transformed and baseline corrected. This yielded a series of 1 D spectra, which display decays of signal amplitudes in dependence of the gradient strength applied. The integrals of the peaks of interest were measured in all experiments and plotted against the gradient strength. A built-in fitting function was employed in order to extract the diffusion coefficient.

The following NMR signals were used for the analysis: benzyl acetate, —(CO)—CH₃, δ=1.96 ppm; (E)-3,7-dimethyl-2,6-octadien-1-ol (geraniol), C═CH—C, δ=5.39 ppm; decanal, CH₃—CH₂—, δ=0.93 ppm; Vertenex®, —C(CH₃)₃, δ=0.92 ppm, hexyl isobutyrate, (CH₃)₂—CH—, δ=1.17 ppm; isobornyl acetate, (CH₃)₂—C—, δ=0.88 ppm; H40-X-(Pn-BuMA)₃₀-(PPEGMA)₃₂, —O—CH₂—CH₂—O—, δ=3.72 ppm.

NMR diffusion spectroscopy has been employed to study encapsulation of small molecules in polymer systems. The mobility of a molecule in solution is defined by its diffusion coefficient. It is inversely proportional to the size of the molecule and hence to its molecular mass. Thus, due to the large difference in mass between fragrance molecules and star block copolymer, the diffusion coefficient of a fragrance molecule in pure water will be different from that entrapped in the copolymer.

All fragrance molecules in pure water exhibit a diffusion coefficient that is very high (around 6×10⁻¹⁰ m²/s) showing unrestricted motion (see Table). Once the fragrance molecules are added to an aqueous solution of the polymer their diffusion behaviour changes dramatically. All molecules experience a decrease in mobility, and the diffusion coefficients are significantly lower than in pure water. Some of the molecules display diffusion coefficients that are reduced by a factor of more than 20, approaching the values of the star block copolymer (around 1×10⁻¹² m²/s). This is a clear indication that the fragrance molecules are entrapped in the polymer. Because of the fact that one can only see a single set of signals for each fragrance molecule in the NMR spectra it can be assumed that the exchange between free and bound states is fast on the NMR time scale, and the observed diffusion coefficients are weighted averages of both states. Other factors such as affinity and the location inside the star block copolymer will also influence the apparent diffusion coefficient.

The following diffusion coefficients of fragrance and amphiphilic star block copolymer molecules were measured in D₂O at 25° C.:

| | | D^{fragrance} | |
|---|---|---|---|
| | D^{fragrance} | (with star block | D^{copolymer} |
| | (D₂O) | copolymer) | (with fragrance) |
| Fragrance molecule | [m²/s] | [m²/s] | [m²/s] |
| Decanal | 5.49 × 10⁻¹⁰ | 2.03 × 10⁻¹¹ | 9.66 × 10⁻¹² |
| Vertenex ® | 5.59 × 10⁻¹⁰ | 2.47 × 10⁻¹¹ | 9.50 × 10⁻¹² |
| Geraniol | 5.98 × 10⁻¹⁰ | 1.74 × 10⁻¹⁰ | 1.13 × 10⁻¹¹ |
| Benzyl acetate | 7.33 × 10⁻¹⁰ | 3.09 × 10⁻¹⁰ | 1.03 × 10⁻¹¹ |
| Isobornyl acetate | 5.86 × 10⁻¹⁰ | 3.50 × 10⁻¹¹ | 9.45 × 10⁻¹² |
| Hexyl isobutyrate | 5.69 × 10⁻¹⁰ | 4.22 × 10⁻¹¹ | 1.03 × 10⁻¹¹ |

### Example 8

Release of Fragrances Followed by Thermogravimetric Analysis (TGA)

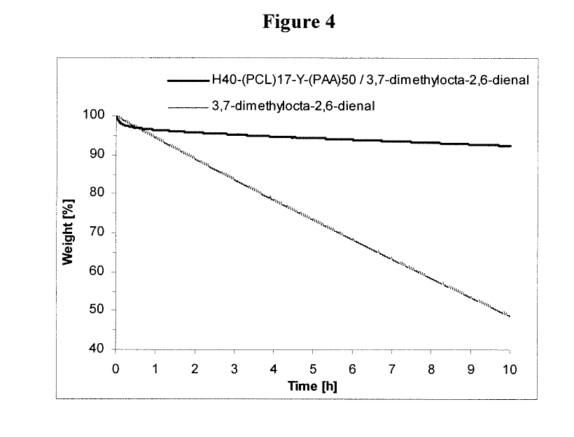

Amphiphilic star block copolymer H40-(PCL)₁₇-Y-(PAA)₅₀ (prepared as described in Example 1.iv) was dried and mixed directly with 3,7-dimethylocta-2,6-dienal (citral) at the mixing ratio of 64/36% (w/w). This sample has been kept at room temperature for at least one day. Then, a mass of approximately 5 mg was introduced into an aluminium oxide crucible and analysed with a Thermogravimetric Analyser (TGA, Mettler Toledo) by recording the isotherms at 30° C. under a constant flow of nitrogen gas (20 mL/min). The analysis was repeated three times and compared to the one of pure citral. FIG. 4 shows the corresponding average curves representing the evolution of the weight (in %) of pure citral and citral/H40-(PCL)₁₇-Y-(PAA)₅₀ mixture as a function of time.

One could observe that the evaporation of citral is strongly slowed down by the presence of the amphiphilic star block copolymer. The isotherm of the release of citral from the H40-(PCL)₁₇-Y-(PAA)₅₀ matrix seems to be composed of two regimes: a fast regime at the beginning of the isotherm followed by a slow one. The isotherm of the release of pure citral is composed of one fast regime.

It is concluded that the amphiphilic star block copolymer of the present invention has a strong retention effect on citral.

In a similar experiment, 40 mg (2% (w/w) of either one of the amphiphilic star block copolymers H40-(PCL)₁₀-Y-(PAA)₇₀ and H40-(PCL)₅₀-Y-(PAA)₅₄ (prepared as described in Example 1.iv) or H40-X-(Pn-BuMA)₃₀-(PPEGMA)₃₂ (prepared as described in Example 2.v) were solubilised in 1.70 g (85% (w/w)) of ethanol. After stirring, 160 mg (8% w/w) of pure water were added and 100 mg (5% (w/w)) of either one of the following fragrance molecules: 4-tert-butyl-1-cyclohexyl acetate (Vertenex®), benzyl acetate, (E)-3,7-dimethyl-2,6-octadienol (geraniol) or decanal. This sample was kept under agitation at room temperature for at least 2 d. In a similar way, a reference sample was prepared using the Boltorn® H40 HBP.

A volume of 10 μL of the sample prepared above was placed in an aluminium oxide crucible and analysed with a Thermogravimetric Analyser (TGA, Mettler Toledo) under a constant flow of nitrogen gas (20 mL/min). The evaporation of the pure fragrance molecule was measured by using the following method that consists in heating the sample from 25 to 50° C. at 5° C./min followed by an isotherm at 50° C. during 115 minutes, then heating from 50° C. to 130° C. at 4° C./min and finally an isotherm at 130° C. during 15 minutes. The analyses were repeated twice and compared to those of the pure fragrance molecules as well as to the Boltorn® H40 HBP reference.

For all four fragrance molecules the measured fragrance evaporation was slower in the 15 presence of either one of the amphiphilic star block copolymers (H40-X-(Pn-BuMA)₃₀-(PPEGMA)₃₂, H40-(PCL)₁₀-Y-(PAA)₇₀, or H40-(PCL)₅₀-Y-(PAA)₅₄), as compared to the Boltorn® H40 HBP reference or as compared to the respective fragrance molecules alone.

To illustrate the effect of the amphiphilic star block copolymers according to the invention on the retention of the fragrance compounds, the weight of the fragrance (in %) was compared after 80 min at 50° C. in the presence of either one of the amphiphilic star block copolymers (H40-X-(Pn-BuMA)₃₀-(PPEGMA)₃₂ or H40-(PCL)₁₀-Y-(PAA)₇₀), the Boltorn® H40 HBP reference, or in the absence of any polymer. The data obtained for the evaporation of the fragrance alone were normalised to account for the polymer content (2% by weight) in the other samples.

Weight [%] after 80 min at 50° C.

| | Fra- | | | H40-X- |
|---|---|---|---|---|
| | grance | Boltorn ® | H40-(PCL)₁₀- | (Pn-BuMA)₃₀- |
| | alone | H40 HBP | Y-(PAA)₇₀ | (PPEGMA)₃₂ |
| Geraniol | 3.5 | 4.35 | 5.89 | 5.65 |
| Decanal | 2.51 | 2.55 | 4.62 | 2.65 |
| Benzyl acetate | 2.00 | 2.64 | 2.46 | 2.65 |
| Vertenex ® | 2.48 | 2.54 | 3.08 | 3.39 |

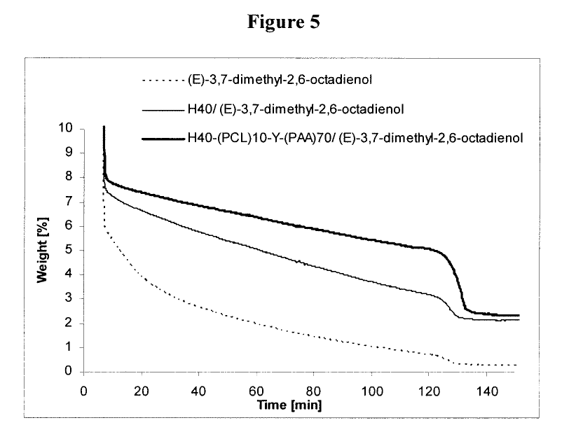

FIG. 5 shows the evaporation (weight in % relative to the initial weight at the beginning of the experiment as a function of time in min) of geraniol alone, geraniol in the presence of Boltorn® H40 HBP and geraniol in the presence of the amphiphilic star block copolymer H40-(PCL)₁₀-Y-(PAA)₇₀. Two regimes appear in the evaporation of fragrance molecules. The first one corresponds to the evaporation of ethanol and the second one to the evaporation of water with geraniol in the presence of the star block copolymer.

The data show that evaporation of the fragrance molecules is significantly slowed down by the presence of any one of the amphiphilic star block copolymers according to the invention. This effect is much more pronounced than in the case with the Boltorn® H40 HBP reference.

### Example 9

Long-Lastingness of the Fragrance Release in the Presence of an Amphiphilic Star Block Copolymer in a Fine Perfumery Application

A model perfume was obtained by mixing equimolecular quantities (0.2 mol) of 15 fragrance compounds with different chemical functionalities (aldehydes, ketones, alcohols, nitriles and esters). The following compounds were weighed in: (Z)-3-hexenol (pipol, 2.00 g), 3,5,5-trimethylhexanal (2.84 g), 2,6-dimethyl-2-heptanol (dimetol, 2.88 g), acetophenone (2.40 g), ethyl (E)-2,4-dimethyl-2-pentenoate (3.12 g), benzyl acetate (3.00 g), jasmonitrile (3.06 g), decanal (3.12g), 4-phenyl-2-butanone (benzylacetone, 2.96 g), 2-pentylcyclopentanol (3.12 g), (E)-3,7-dimethyl-2,6-octadienol (geraniol, 3.08 g), 4-cyclohexyl-2-methyl-2-butanol (3.40 g), 10-undecenal (3.36 g), 4-tert-butyl-1-cyclohexyl acetate (Vertenex®), 3.96 g), allyl 3-cyclohexylpropanoate (3.92 g).

Amphiphilic star block copolymer H40-(PCL)₁₀-Y-(PAA)₇₀ (prepared as described in Example 1.iv) 40 mg (2% (w/w)) was solubilised in 1.70 g (85% (w/w)) of ethanol. After stirring, 160 mg of water were added and 100 mg (5% (w/w)) of the model perfume described above. The sample was kept under agitation at room temperature for at least 3 d. A total of 2 μL of the sample was then placed in a headspace sampling cell (160 mL) thermostatted at 25° C. and exposed to a constant air flow of 200 mL/min, respectively. The air was filtered through active charcoal and aspirated through a saturated solution of NaCl. The volatiles were continuously adsorbed onto 100 mg Tenax® TA cartridges, which were changed after t=3.5, 4.5, 6, 8, 10, 13, 16, 20, 30, 45, and 60 min. The cartridges were desorbed thermally in a Perkin Elmer TurboMatrix ATD desorber and the volatiles analysed with a Carlo Erba MFC 500 gas chromatograph equipped with a FID detector. The analyses were effected using a J&W Scientific DB capillary column (30 m×0.45 mm i.d., film thickness 0.42 μm) from 70° C. to 130° C. (at 3° C./min) then to 260° C. at 35° C./min. Injection temperature was 240° C., detector temperature was 260° C. Headspace concentrations (in ng/L) were obtained by external standard calibration of the corresponding fragrance molecules using six different concentrations in ethanol. 0.2 μL of each calibration solution were injected onto Tenax® TA cartridges, which were desorbed under the same conditions described before. The results are the average of two measurements.

The same experiment was repeated using 2 μL of the model perfume described above without the amphiphilic star block copolymer as a reference sample in order to compare the long-lastingness of the fragrance evaporation in the presence or absence of the amphiphilic star block copolymer.

For the comparison of the data, the time of reaching an arbitrarily chosen headspace concentration of 50 ng/L was determined. The following data was obtained:

Time required to reach a headspace
concentration of 50 ng/L [min]

| | no | H40-(PCL)₁₀- |
|---|---|---|
| Name of fragrance compound | copolymer | Y-(PAA)₇₀ |
| Pipol | 5.0 | 5.2 |
| 3,5,5-Trimethylhexanal | 8.0 | 9.3 |
| Dimetol | 7.8 | 10.0 |
| Acetophenone | 6.0 | 10.3 |
| Ethyl (E)-2,4-dimethyl-2-pentenoate | *) | 6.2 |
| Benzyl acetate | 9.7 | 17 |
| Jasmonitrile | 11.6 | 13.6 |
| Decanal | 16.7 | 16.7 |
| Benzylacetone | 15.5 | 22.0 |
| 2-Pentylcyclopentanol | 30.6 | 37.8 |
| Geraniol | 42.0 | 45.6 |
| 4-Cyclohexyl-2-memyl-2-butanol | 35.8 | 42.6 |
| 10-Undecenal | 29.2 | 30.6 |
| Vertenex ® | 24.6 | 31.0 |
| Allyl 3-cyclohexylpropanoate | 31.9 | 44.0 |

*) headspace concentration always below 50 ng/L.

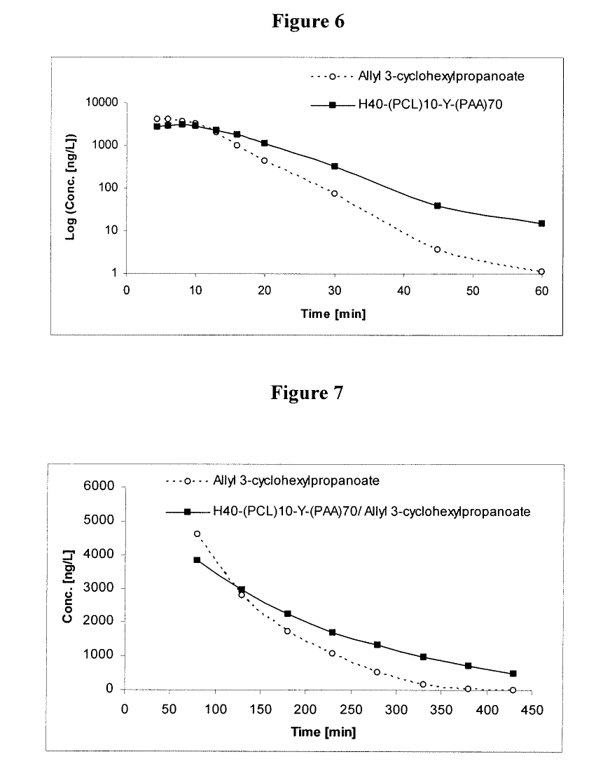

FIG. 6 represents the evaporation profile of one of the compounds (allyl 3-cyclohexylpropanoate) contained in the perfume, chosen as an example to show the long-lastingness of the fragrance compounds in the presence of the amphiphilic star block copolymer H40-(PCL)₁₀-Y-(PAA)₇₀.

In general, the intensity of the fragrance molecules was found to be higher when an amphiphilic star block copolymer according to the invention was present.

### Example 10

Long-Lastingness of the Fragrance Release in the Presence of an Amphiphilic Star Block Copolymer in a Fabric Softener Application

The use of the present invention's amphiphilic star block copolymers has been tested for the controlled release of fragrances in a fabric softener application. A fabric softener base with the following composition has been prepared:
Stepantex® VK90 or VHR90 (origin: Stepan) 16.5% by weight
Calcium chloride 0.2% by weight
Water 83.3% by weight
   The performance of a mixture of fragrance molecules in a fabric softener was determined by comparing the performance of the fragrance evaporation in the presence and absence of an amphiphilic star block copolymer. The experiments were carried out as follows:

### 10.i Fabric Softening Procedure Using Amphiphilic Star Block Copolymer H40-(PCL)₁₀-Y-(PAA)₇₀

A solution of equimolar amounts (0.45 mmol) of 4-phenyl-2-butanone (benzylacetone, 63.8 mg), allyl 3-cyclohexylpropanoate (86 mg), 4-cyclohexyl-2-methyl-2-butanol (78.7 mg) and benzyl acetate (69.3 mg) in 10 mL of ethanol was prepared. 3.30 mL of this solution were added to 40 mg (1.33.10⁻⁴ mmol) of amphiphilic star block copolymer H40-(PCL)₁₀-Y-(PAA)₇₀ and stirred for one day. A total of 1.80 g of the above described fabric softener base was weighed into two small vials, respectively. Then, 1 mL of the solution containing the fragrance molecules and the polymer was added to one of the samples, and 1 mL of the solution containing the fragrances and no polymer to the other. Both vials were closed and left under agitation at room temperature for 4 d. The samples were then dispersed in a beaker with 600 mL of demineralised cold tap water, respectively. One cotton towel (EMPA cotton test cloth Nr. 221, origin: Eidgenössische Materialprüfanstalt (EMPA), pre-washed with an unperfumed detergent powder and cut to ca. 12×12 cm sheets) was added to each beaker and agitated manually for 3 min, left standing for 2 min, then wrung out by hand and weighed to obtain a constant quantity of residual water. The two towels (one with the amphiphilic star block copolymer and one without) were analysed immediately after treatment with the softener. For the measurements, one towel was put into an headspace sampling cell (160 mL) thermostatted at 25° C. and exposed to a constant air flow of 200 mL/min, respectively. The air was filtered through activated carbon and aspirated through a saturated solution of NaCl. The headspace system was equilibrated for 75 min, then the volatiles were adsorbed during 5 min on a clean Tenax® cartridge. The sampling was repeated 7 times every 50 min. The cartridges were desorbed on a Perkin Elmer TurboMatrix ATD desorber as described above (Example 9).

FIG. 7 shows a typical example for the release of one of the fragrance molecules (allyl 3-cyclohexylpropanoate) in the presence or absence of H40-(PCL)₁₀-Y-(PAA)₇₀.

At the beginning of the measurement higher headspace concentration were measured in the absence of the amphiphilic star block copolymer. Nevertheless, at the end of the experiment all headspace concentrations were higher in the presence of the amphiphilic star block copolymer thus showing the desired long-lastingness of the fragrance release. After 430 min the following headspace concentration were obtained:

| | Headspace concentration | Headspace concentration |
|---|---|---|
| | in the absence of | in the presence of H40- |
| | H40-(PCL)₁₀-Y-(PAA)₇₀ | (PCL)₁₀-Y-(PAA)₇₀ |
| | [ng/L] | [ng/L] |
| Benzyl acetate | 0.0 | 34.7 |
| Benzylacetone | 74.4 | 130.6 |
| 4-cyclohexyl-2- | 69.7 | 445.8 |
| methyl-2-butanol | | |
| Allyl-3- | 10.3 | 498.1 |
| cyclohexyl- | | |
| propanoate | | |

### 10.ii Fabric Softening Procedure Using Amphiphilic Star Block Copolymer H40-X-(Pn-BuMA)₃₀-(PPEGMA)₃₂

The experiment was carried out as described above using amphiphilic star block copolymer H40-X-(Pn-BuMA)₃₀-(PPEGMA)₃₂ instead of H40-(PCL)₁₀-Y-(PAA)₇₀ . The headspace system was equilibrated for 15 min, and the volatiles were adsorbed during 5 min. The sampling was repeated 7 times every 50 min.

At the beginning of the measurement higher headspace concentration were measured in the absence of the amphiphilic star block copolymer. Nevertheless, at the end of the experiment all headspace concentrations were higher in the presence of the amphiphilic star block copolymer thus showing the desired long-lastingness of the fragrance release. After 370 min the following headspace concentration were obtained:

| | Headspace concentration | Headspace concentration |
|---|---|---|
| | in the absence of | in the presence of |
| | H40-X-(Pn—BuMA)₃₀- | H40-X-(Pn—BuMA)₃₀- |
| | (PPEGMA)₃₂ [ng/L] | (PPEGMA)₃₂ [ng/L] |
| Benzyl acetate | 6.3 | 82.1 |
| Benzylacetone | 277.3 | 298.2 |
| 4-cyclohexyl-2- | 375.2 | 837.2 |
| methyl-2-butanol | | |
| Allyl-3- | 46.8 | 588.1 |
| cyclohexyl- | | |
| propanoate | | |

What is claimed is:

1. A block copolymer compound comprising the general formula (I) 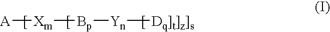
wherein
A is a core having s functionalities; s multiplied by z defines the number of arms of the copolymer, whereby the product of s*z>6;
Xm and Yn are, independently of each other, a linear or branched linker moiety with m or n, independently of each other, being 0 or 1, which is, once grafted to the core, suitable as a starting point for at least one polymerisation reaction;
z and t are the number of branching provided by each of the linker moieties X and Y, respectively, with z and t being, independently, in the range of 1-10;
B is a polymerised moiety having a calculated Hansen solubility parameter of ≦25, which is covalently linked to a functionality of A or to a functionality of X, with p being the average number of polymerised B moieties, p is in the range of 3-300;
D is a polymerised moiety having a Hansen solubility parameter of >25 with q being the average number of polymerised D moieties, q is in the range of 3-300.

2. The copolymer according to claim 1, which has a mean diameter of 2-150 nm.

3. The copolymer according to claim 1, which has a molecular weight Mn of >100,000 g/mol.

4. The copolymer according to claim 1, further comprising at least one lipophilic functional agent encapsulated in or associated to the copolymer, the functional agent being selected from the group consisting of a flavour, a fragrance, a drug, an agrochemical, a dye, and mixtures thereof.

5. The copolymer according to claim 1, wherein block B is selected from the group consisting of poly(methyl methacrylate), poly(methyl acrylate), poly(n-butyl methacrylate), poly(n-butyl acrylate), polylactides, polycaprolactone such as poly(ε-caprolactone), polypropylene glycol, polyanhydrides, polysiloxanes, polyphosphazenes, polyazolines and combinations thereof.

6. The copolymer according to claim 1, wherein block D is selected from the group consisting of poly(methacrylic acid), poly(acrylic acid), poly(dimethyl aminoethyl methacrylate), poly(trimethylaminoethyl methacrylate), poly(trimethylaminoethyl acrylate), poly(trimethylammoniumethyl methacrylate salts), poly(hydroxyethyl methacrylate), poly(methylether diethyleneglycol methacrylate), poly(ethylene oxide), poly(vinylpyrrolidone), poly(polyethylene glycol acrylate), poly(polyethylene glycol methacrylate), polyaminoacids, polyacrylonitriles, poly(ethylene imine), and, polyoxazoline, and combinations thereof.

7. A nano-capsule consisting essentially of the block polymer according to claim 1.

8. A block copolymer, suitable for encapsulation of hydrophobic functional agents, the block copolymer comprising, in this order,
a central, lipophilic or hydrophilic, multifunctional core (A),
a lipophilic block (B), and,
a hydrophilic block (D);
and, optionally, one or more linker molecules between the core and the lipophilic block (X) and/or between the lipophilic block and the hydrophilic block (Y).

9. A perfumed product comprising the block copolymer of claim 1.

10. A process for manufacturing a block copolymer comprising the steps of
providing a core (A) having s functionalities, with s>5,
optionally, linking the functionalities of the core to a linker moiety (X),
polymerising a hydrophobic block (B) onto the functionality of the core (A), or, if present, onto the linker moiety (X),
optionally, linking a further linker moiety (Y) onto the hydrophobic block (B), and,
polymerising a hydrophilic block (D) onto the functionality of the hydrophobic block (B) or, if present, onto the further linker moiety (Y), or, alternatively,
polymerising a second hydrophobic block onto the hydrophobic block (B) or, if present, onto the further linker moiety (Y), followed by chemically transforming the second hydrophobic block into a hydrophilic block (D).
